SEQUENCE LISTING (6 sequences)

SEQ ID NO: 1 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 2 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 3 - protein, 626 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 4 - DNA, 2699 bp, Homo sapiens
ggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaaaaaaaaaaaaaaaaaaaaaaaaaaaaaaa

SEQ ID NO: 5 - DNA, 3447 bp, Homo sapiens
ggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaaaaaaaaaa

SEQ ID NO: 6 - DNA, 5635 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

(19) United States
(12) Patent Application Publication
(10) Pub. No.: US 2010/0204248 A1
(43) Pub. Date: Aug. 12, 2010

(54) MEDICAMENTS AND METHODS FOR INHIBITION OF NON-OCULAR SCARRING

(75) Inventors: Mark William James Ferguson, Derbyshire (GB); Nicholas Occleston, Manchester (GB); Sharon O'Kane, Manchester (GB); Nicholas Goldspink, Manchester (GB); Kerry Nield, Manchester (GB)

Correspondence Address: FOLEY AND LARDNER LLP;SUITE 500, 3000 K STREET NW, WASHINGTON, DC, 20007, US

(73) Assignee: Renovo Limited

(21) Appl. No.: 12/671,005

(22) PCT Filed: Jul. 31, 2008
(86) PCT No.: PCT/GB08/02609

§ 371 Date: Jan. 27, 2010

(30) Foreign Application Priority Data
Aug. 1, 2007 (GB) 0714934.7

(51) Int. Cl. A61K 31/52; C07D 473/38; A61P 17/02

(52) U.S. Cl. 514/263.3; 544/265

(57) ABSTRACT

Provided is the use of an agonist of a member of the nuclear hormone receptor NR4A subgroup in the manufacture of a medicament for the prevention, reduction or inhibition of scarring in a non-ocular tissue. Also provided is a method of preventing, reducing or inhibiting scarring in a non-ocular tissue, the method comprising administering a therapeutically effective amount of an agonist of a member of the nuclear hormone receptor NR4A subgroup to a patient in need of such prevention, reduction or inhibition. The NR4A agonist may be 6-mercaptopurine. The medicaments and methods may preferably be used to prevent, reduce or inhibit scarring in the skin. The medicaments and methods of the invention may be used to accelerate healing of wounds.

The present invention relates to the manufacture of medicaments for the prevention, reduction or inhibition of scarring in non-ocular tissues. The invention also provides methods for the prevention, reduction or inhibition of scarring in non-ocular tissues. The medicaments or methods of the invention may be used for the prevention, reduction or inhibition of scarring associated with wounds of non-ocular tissues, or of scarring associated with fibrotic disorders in non-ocular tissues.

Clinical approaches to wound management will generally depend on the outcome that it is desired to achieve. This outcome may, for example, be considered with reference to the degree of scarring occurring, or with reference to the speed at which a wound heals. In management of some wounds control of the degree of scarring that occurs is of primary importance, while increasing the speed of wound healing is of much lesser importance. In management of other wounds increasing the speed of wound healing is of primary importance, while controlling the degree of scarring occurring is of much lesser importance. However, in many cases it will be beneficial to be able to control the degree of scarring that occurs while also increasing the speed of wound healing. This may be particularly important in circumstances in which the continued presence of a wound in a tissue or organ will be associated with disadvantages such as pain, increased risk of infection, or mechanical failure.

Many different processes are at work during the scarring response, and much research has been conducted into discovering what mediates these processes, and how they interact with each other to produce the final outcome.

The scarring response is common throughout all adult mammals. Scarring may result from healing of a wound, or through the deposition of scar tissue associated with fibrotic disorders. The scarring response results in the formation of fibrotic tissue termed a “scar”. A scar may be defined as “fibrous connective tissue that forms at the site of injury or disease in any tissue of the body”. Generally, the scarring response is conserved between the majority of tissues or organs of the body, but in certain cases the response differs from tissue to tissue.

In the case of a scar that results from healing of a wound, the scar constitutes the structure produced as a result of the reparative response. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue. The imperative for wounds to heal quickly is often thought to be linked to “bad” scarring, in which the scar produced is notably different from unwounded tissue, while slower healing may be associated with “good” scarring, in which the difference between the scar and unwounded tissue is relatively small.

Although scarring may most frequently occur on healing of a wound, similar disturbances of the extracellular matrix may also give rise to scarring associated with a number of medical conditions known as fibrotic disorders. In these disorders excessive fibrosis leads to pathological derangement and malfunctioning of tissue. Scars associated with fibrotic disorders are characterised by the accumulation of fibrous tissue in an abnormal fashion within the diseased area. Accumulation of such fibrous tissues may result from a variety of disease processes, all of which are capable of leading to the production of a scar.

Fibrotic disorders are usually chronic. Examples of fibrotic disorders include cirrhosis of the liver, liver fibrosis, glomerulonephritis, pulmonary fibrosis, chronic obstructive pulmonary disease, myocardial fibrosis, fibrosis following myocardial infarction, arthritis and adhesions e.g. in the digestive tract, abdomen, pelvis, tendon and spine. If left untreated, the pathological effects of scarring associated with fibrotic disorders may lead to organ failure, and ultimately to death.

The biological and pathological processes underlying the development of scars associated with fibrotic disorders are sufficiently similar to those involved in the formation of scars resulting from healing of a wound that those compounds that may be used to prevent, reduce or inhibit scarring associated with one form will generally be similarly effective in the other form of scarring.

Scars, whether produced as a result of wounds or of fibrotic disorders, are composed of connective tissue. In the case of wounds this material is deposited during the healing process, whereas in fibrotic disorders it occurs as a result of the disease process. A scar may comprise connective tissue that has an abnormal organisation, as is frequently observed in scars of the skin. Alternatively or additionally, a scar may comprise connective tissue that is present in an abnormally increased amount. Most scars consist of both abnormally organised and excess connective tissue, as described further below.

The abnormal structure of scars may be observed with reference to both their internal structure (which may be determined by means of microscopic analysis) and their external appearance (which may be assessed macroscopically).

In connective tissues, such as the skin, extracellular matrix (ECM) molecules comprise the major structural component of both “normal” (unwounded) and scarred tissues. In normal skin these molecules form fibres which, when viewed microscopically, have a characteristic random arrangement that is commonly referred to as “basket-weave”. This basket-weave arrangement is disrupted in scars. Fibres in scars exhibit a marked degree of alignment with each other as compared to the random arrangement of fibres in normal skin. In general the fibres observed within scars are also of smaller diameter than those seen in normal skin. Both the size and arrangement of ECM may contribute to the scars altered mechanical properties, most notably increased stiffness, when compared with normal skin.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the cause of the scar, be it either a wound or a fibrotic disorder, has passed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

Scars occur at many body sites, and the effects of scarring at these sites will generally be related to loss or disruption of function in the scarred area. Some of the disadvantages associated with scarring of the skin have been discussed above. Scarring of the internal organs may lead to the formation of strictures and adhesions that significantly or totally impair function of the organ in question. Scarring of tendons and ligaments may cause lasting damage to these organs, and thereby reduce the motility or function of associated joints. Scarring associated with blood vessels, and particularly the valves of the heart, may occur after injury or surgery. Scarring of blood vessels may lead to restenosis, which causes a narrowing of the blood vessel and thus reduces the flow of blood through the scarred area. Scarring in the central and peripheral nervous system may prevent transmission along the nerve and may prevent or reduce reconnection of damaged nerve tissue, and/or functional neuronal transmission.

The effects outlined above may all arise as a result of the normal progression of the wound healing response (in the case of scars that result from healing of a wound). There are, however, many ways in which the scarring response may be abnormally altered; and these are frequently associated with even more damaging effects resulting from the production of abnormal excessive scarring (commonly referred to as pathological scarring). There are a number of methods by which pathological scarring may be differentiated from severe scarring resulting from the normal healing response. These include histological differences in the scars produced, as well as genetic markers that may indicate a disposition to pathological scarring. An individual's history of pathological or non-pathological scarring remains one of the most effective predictors of the likelihood of future incidences of pathological scarring. The most frequent and important classes of pathological scarring include hypertrophic scarring and keloid scarring.

Whilst much of the present specification concentrates primarily on the effects of scarring in man (whether scarring that results from healing of a wound, or scarring associated with fibrotic disorders), it will be appreciated that many aspects of the scarring response are conserved between most species of animals. Thus, the problems outlined above are also applicable to non-human animals, and particularly veterinary or domestic animals (e.g. horses, cattle, dogs, cats etc). By way of example, it is well known that adhesions resulting from the inappropriate healing of abdominal wounds constitute a major reason for the veterinary destruction of horses (particularly race horses). Similarly the tendons and ligaments of domestic or veterinary animals are also frequently subject to injury, and healing of these injuries may also lead to scarring associated with increased animal mortality.

Although the ill effects of scarring (either resulting from normal or aberrant wound healing, or associated with fibrotic disorders) are well known there remains a lack of effective therapies able to reduce these effects. In the light of this absence it must be recognised that there exists a strongly felt need to provide medicaments and treatments that are able to prevent, reduce or inhibit scar formation, whether resulting from healing of a wound, or associated with fibrotic disorders.

The nuclear hormone receptors (NRs) are a superfamily of transcription factors that act as intracellular signalling molecules, and generally effect gene expression in response to the presence of their ligand binding partners. The NR superfamily can be classified either on the basis of their mechanism of action, or based on their sequence homology. Using classification based on sequence homology (Nuclear Receptors Nomenclature Committee, Cell 1999), the NR4A subgroup represents a group of “orphan” receptors consisting of: NR4A1 (also known as GFRP1, HMR, MGC9485, N10, NAK-1, NGFIB, NP10, NUR77, TR3), NR4A2 (HZF-3, NOT, NURR1, RNR1, TINUR) and NR4A3 (CHN, CSMF, MINOR, NOR1, TEC). Amino acid sequences of these proteins, as well as nucleic acid sequences encoding the proteins, are provided elsewhere in the specification as follows: Sequence ID No. 1—amino acid sequence of NR4A1; Sequence ID No. 2—amino acid sequence of NR4A2; Sequence ID No. 3—amino acid sequence of NR4A3; Sequence ID No. 4—nucleic acid sequence encoding NR4A1; Sequence ID No. 5—nucleic acid sequence encoding NR4A2; Sequence ID No. 6—nucleic acid sequence encoding NR4A3. The NR4A subgroup are classified as early response genes that are induced by a range of physiological signals including fatty acids, prostaglandins, growth factors, peptide hormones, and physical stimuli such as shear stress or magnetic fields.

The members of the NR4A subgroup are well conserved in the DNA binding domain (−91-95%) and the C-terminal ligand-binding domain (−60%), although the NR4A family are thought to activate gene expression in a ligand-independent manner. In the “classical” model of NR regulation, a hydrophobic cleft in the ligand-binding domain (LBD) recruits co-factors that function as co-activators or co-repressors of transcription. However, the NR4A receptors encode unusual and atypical LBDs, which do not appear to function in this manner.

The NR4A subfamily has been extensively investigated in the context of inflammation, and it is known that all members of this subfamily are rapidly induced in macrophages following lipopolysaccharide (LPS) and cytokine stimulation (i.e. IFNγ, TNFα).

It is an aim of certain aspects of the present invention to provide medicaments suitable for the prevention and/or reduction and/or inhibition of scarring in non-ocular tissues. It is an aim of further aspects of the present invention to provide methods of treatment suitable for use in the prevention, and/or reduction, and/or inhibition of scarring in non-ocular tissues. It is an aim of certain embodiments of the invention to provide medicaments suitable for the prevention and/or treatment of scarring that results from healing of a wound in non-ocular tissues. It is an aim of certain embodiments of the invention to provide medicaments suitable for the prevention and/or treatment of scarring associated with fibrotic disorders in non-ocular tissues. It is an aim of certain embodiments of the invention to provide methods of treatment suitable for use in the prevention and/or treatment of scarring that results from the healing of a wound in non-ocular tissues. It is an aim of further embodiments of the invention to provide methods of treatment suitable for use in the prevention and/or treatment of scarring associated with fibrotic disorders in non-ocular tissues. It is an aim of certain embodiments of the invention to provide medicaments and/or methods of treatment that accelerate the healing of wounds. The medicaments and/or methods of the invention may constitute alternatives to those provided by the prior art. However, it is preferred that medicaments and/or methods of treatment provided by the invention may constitute improvements over the prior art.

According to a first aspect of the present invention there is provided the use of an agonist of a member of the nuclear hormone receptor NR4A subgroup in the manufacture of a medicament for the prevention, reduction or inhibition of scarring in a non-ocular tissue. This first aspect of the invention also provides an agonist of a member of the nuclear hormone receptor NR4A subgroup for use as a medicament for the prevention, reduction or inhibition of scarring in a non-ocular tissue. The medicament may be for localised administration at the site where scarring is to be prevented, reduced or inhibited. The medicament may be a topical medicament for application at a site where scarring is to be prevented, reduced or inhibited. The medicament may preferably be for use at a wound, or at a site where a wound is to be formed.

In a second aspect of the invention there is provided a method of preventing, reducing or inhibiting scarring in a non-ocular tissue, the method comprising administering a therapeutically effective amount of an agonist of a member of the nuclear hormone receptor NR4A subgroup, to a patient in need of such prevention, reduction or inhibition. The NR4A agonist, may preferably be administered to the site where scarring is to be prevented, reduced or inhibited. The site may preferably be a wound in a non-ocular tissue, or a site where a wound in a non-ocular tissue is to be formed.

It may be preferred that the medicaments or methods of the invention utilise 6-mercaptopurine as a suitable NR4A agonist. 6-mercaptopurine may be used as the sole NR4A agonist provided, or may be used in combination with one (or more) further NR4A agonists.

The present invention is based on the inventors' new and surprising finding that an agonist of a member of the nuclear hormone receptor NR4A subgroup, such as 6-mercaptopurine, may be used in the prevention, reduction or inhibition of scarring. 6-mercaptopurine is known to act as an agonist of all three members of the NR4A subgroup i.e NR4A1, NR4A2 and NR4A3 (Wansa et. al. 2003). The inhibition of scarring achieved is apparent with reference to both the macroscopic appearance of a treated scar, and the microscopic appearance of the scar's inner structure. There are no previous reports that would lead the skilled person to believe that NR4A agonists, such as 6-mercaptopurine, may be used to effectively prevent, reduce or inhibit scarring.

The finding that NR4A agonists, such as 6-mercaptopurine, may be used to prevent, reduce or inhibit scarring provides the foundation for new medicaments and methods that may be used in the treatment, management or improvement of scarring. Furthermore, the inventors' finding that NR4A agonists, such as 6-mercaptopurine, may be used in the prevention, reduction or inhibition of scarring offers the prospect that improved medicaments and methods having greater efficacy, may be made available for the treatment or management of scarring.

As well as being agonists of NR4A, 6-mercaptopurine is also known to act as a antimetabolic agent, or antimetabolite. The antimetabolic effects of 6-mercaptopurine is thought to be achieved as a result of its similarity to purines, which are essential for DNA replication. Presence of 6-mercaptopurine in cells inhibits the normal incorporation of purines into DNA, thus preventing cell division. It has previously been suggested that antimetabolites may be administered to wounds in the eye in order to inhibit fibroblast proliferation, and thereby reduce scarring. However, the use of antimetabolites in the eye in this manner is associated with inhibition of the wound healing response. This is because the antimetabolites effectively “poison” cells, such as fibroblasts, at the wound site. This reduces accumulation of extracellular matrix components that may otherwise be deposited by such cells, but also prevents the cells contributing beneficially to the wound healing response. The inhibition of wound healing observed when antimetabolites are provided to eye wounds gives rise to various complications such as leakage from the improperly healed wound site and defects in re-epithelialisation (caused by toxic effects of antimetabolites on epithelial cells).

The ability of antimetabolites to reduce scarring in the eye would not necessarily be taken as indicating that the biological effect of these agents would be the same in other, non-ocular, sites. However, as a result of the mechanisms by which these agents achieve their anti-scarring effect in the eye (i.e. by poisoning cells that deposit extracellular matrix components during the healing response), it would be believed by those skilled in the art that any amount of an antimetabolite capable of reducing scarring would also inhibit wound healing.

The inhibited wound healing observed on use of 6-mercaptopurine in the eye has meant that those skilled in the art would not believe that this agent would be suitable for use in non-ocular tissues. Complications that are undesirable in the eye, but considered acceptable in exchange for reduced scarring, may be more deeply damaging in non-ocular tissues, such as the skin. While the eye is kept clean, moistened and generally sterile through the actions of tears, other tissues or organs may be placed at much increased risk of infection if their wounds are subject to impaired healing. This is particularly so for the skin, which serves as the barrier between the body and the outside environment. Here, inhibition of wound healing often leads to infection (as a result of greater exposure to pathogenic organisms) and causes damaging dehydration of the injured site. The lack of mechanical strength that occurs due to the extended duration of wounds subject to inhibited wound healing also represents a marked problem in tissues such as the skin (where the ability of the tissue to bear up to mechanical stresses is a highly important function of the tissue).

The present invention is based on the inventors' surprising finding that NR4A agonists, such as 6-mercaptopurine, can be used to prevent, reduce or inhibit scarring in non-ocular tissues. Furthermore, the inventors have very surprisingly found that such anti-scarring effects in non-ocular tissues may be achieved without inhibiting wound healing. In fact, the inventors have found that NR4A agonists, such as 6-mercaptopurine, are able to inhibit scarring in non-ocular tissues, while at the same time accelerating wound healing.

It will be appreciated that the ability to both inhibit scarring and accelerate healing of a wound to which the medicaments (or methods) of the invention are provided is of great advantage. Not only may the deleterious effects of scarring be reduced or avoided, but this can be achieved with a reduced period of post-wounding care (since the period required for wound closure to be completed may be reduced) and with a reduced likelihood of complications that may be associated with open wounds (including risk of infection, duration of pain and dehydration).

The scarring, prevention, reduction or inhibition of which is to be achieved by the medicaments or methods of the invention, may be scarring that results from healing of a wound in a non-ocular tissue, or, additionally or alternatively, may be scarring associated with a fibrotic disorder in a non-ocular tissue. It may generally be preferred that the scarring to be prevented, reduced or inhibited is scarring that results from the healing of a wound.

Preferably the scarring that is to be prevented, reduced or inhibited may be non-pathological scarring, that is to say scarring that is not associated with the production of pathological scars such as keloids or hypertrophic scars. Non-pathological scarring to be prevented, reduced or inhibited using the medicaments or methods of the invention may even more preferably be non-pathological scarring that results from the healing of a wound.

The inventors believe that the prevention, reduction or inhibition of scarring using an NR4A agonist, such as 6-mercaptopurine, can be effected at any body site other than the eye and in any non-ocular tissue or organ. However, the skin represents a preferred organ in which scarring may be prevented, reduced or inhibited utilising the medicaments or methods of the invention. Such scarring of the skin may result from healing of a skin wound and/or may be associated with a fibrotic disorder involving the skin.

Scarring resulting from the healing of skin wounds represents a form of scarring that may particularly benefit from prevention, reduction or treatment in accordance with the present invention, and with the medicaments or methods of the present invention. Accordingly, it will also be recognised that skin wounds, or sites where skin wounds are to be formed, may beneficially be treated using the medicaments or methods of the invention.

NR4A agonists, such as 6-mercaptopurine, may preferably be administered to a site that may be associated with scarring (for the present purposes a site where scarring has already occurred, is occurring, or may be expected to occur). For example, NR4A agonists, such as 6-mercaptopurine, may be provided to a patient's wound that would otherwise be likely to give rise to a scar, or may be provided to a site where an increased likelihood of fibrosis has been identified.

NR4A agonists, such as 6-mercaptopurine, may be administered to an existing scar to prevent the further progression of scarring. Administration of NR4A agonists, such as 6-mercaptopurine, to an existing scar may also reduce the level of scarring associated with the existing scar. It will thus be appreciated that NR4A agonists, such as 6-mercaptopurine, may be administered to a site of a fibrotic disorder in order to prevent further scarring, and/or to reduce scarring that has already occurred in association with the fibrotic disorder. Preferred routes of administration that may be used in accordance with all of the embodiments considered above include topical administration, localised administration and particularly localised injection (such as intradermal injection) of suitable active agents.

Without wishing to be bound by any hypothesis, the inventors believe that the ability of the medicaments and methods of the invention to accelerate wound healing may arise as a result of their promotion of wound contraction and/or promotion of re-epithelialisation.

The ability to promote wound contraction is clinically advantageous since it may reduce the size of wounds. This ability to amplify the natural wound contraction response to bring about therapeutic benefits should be differentiated from pathological contraction that may occur in conditions such as hypertrophic scarring. It will be appreciate that wounds in which contraction has been promoted to bring about a reduced size will be more amenable to procedures intended to bring about wound closure, such as grafting or suturing of wounds. Furthermore, smaller wounds produced on promotion of contraction may heal faster than wounds the contraction of which has not been promoted.

The ability to promote re-epithelialisation of wounds is advantageous since it allows wounds to close more rapidly, and hastens the formation of a functional epithelial barrier. This is of great importance in a number of tissues (including graft donor sites and sites subject to dermabrasion or chemical peels) and helps prevent infection and dehydration.

Materials or methods of the invention capable of accelerating wound healing may be of benefit in the treatment of acute or chronic wounds.

Particular acute wounds that may benefit from accelerated healing provided by the medicaments or methods of the invention include surgical wounds (and particularly those associated with cosmetic procedures and/or grafting procedures), wounds resulting from skin peels (or other procedures leading to the production of partial-thickness wounds), and acute wounds with a heightened disposition to chronic wound formation. Wounds that may be expected to have a predisposition to chronic wound formation include pre-tibial lacerations, and wounds of elderly patients, diabetic patients and patients with polypharmacy.

Preferred chronic wounds that may be treated with the medicaments or methods of the invention in order to accelerate their healing include ulcers such as diabetic ulcers, decubitus ulcers, and venous ulcers.

Various terms that are used in the present disclosure to describe the invention will now be explained further. The definitions and guidance provided below may be expanded on elsewhere in the specification as appropriate, and as the context requires.

### “Agonist of a Member of the Nuclear Hormone Receptor NR4A Subgroup”

Receptor agonists are compounds that are able to directly trigger a response in the cell via activation of a cell receptor. For the purpose of the present disclosure an “agonist of a member of the nuclear hormone receptor NR4A subgroup” (otherwise referred to as an “NR4A agonist” for purposes of brevity) should be taken, except for where the context requires otherwise, to encompass any agonist of any member, or number of members, of the NR4A subgroup, provided that the agonist is capable of inhibiting scarring. Preferred means by which such inhibition of scarring may be assessed (and quantified if required) are considered elsewhere in the specification, and include the use of macroscopic and/or microscopic scarring visual analogue scales.

In humans, the NR4A subgroup of nuclear hormone receptors comprises three members: NR4A1; NR4A2; and NR4A3. These members all have alternative nomenclature, as discussed in the introduction to this specification. The NR4A subgroup are so-called “orphan receptors” in that their endogenous ligands are not known, or at least are not generally agreed upon.

For the purposes of the present disclosure, an “agonist of a member of the nuclear hormone receptor NR4A subgroup” may be taken to comprise any agonist that is capable of activating gene transcription by one, or more, members of the NR4A subgroup. A preferred agonist may be one that is able to activate gene transcription by one, or both, of NR4A1 and NR4A3.

Various compounds that may be used as agonists of a member of the NR4A sub-group are known from the prior art, and suitable agonists that may be used in the medicaments and methods of the invention will be apparent to the skilled person. As mentioned elsewhere in the specification, 6-mercaptopurine represents a preferred NR4A agonist for use in the medicaments and methods of the invention. The monohydrate form of 6-mercaptopurine represents a preferred form that may be used in the medicaments or methods of the invention, however, it would be appreciated that any therapeutically effective hydrate or salt of 6-mercaptopurine may be used.

6-mercaptopurine may exist in a number of tautomeric forms, and the skilled person will appreciate that any of these forms, or any mixture of these forms, may be used in the medicaments and methods of the invention. Merely by way of example, 3,7-dihydropurine-6-thione represents a preferred tautomer of 6-mercaptopurine that may be used in the medicaments or methods of the invention.

In the event that the relevant art provides no indication as to whether or not a compound of interest is an NR4A agonist, the ability of the compound to activate gene transcription by one or more members of the NR4A subgroup may be investigated using any suitable assays known to the skilled person. These include, but are not limited to, assays described in:

i) Zetterstrom R. H., Solomin L., Mitsaidis T., Olson L., Perimam T., Mol. Endo., 1996, 10, 1656-1666; and in

### ii) Ordentlich P., Yan Y., Zhou S., and Heyman R., Journal of Biological Chemistry., 2003, 278, 24791-24799.

The disclosures of these documents, in particular insofar as they relate to assays, by which compounds of interest may be assessed for NR4A agonist activity, are incorporated by reference.

Briefly, the assay described in i) utilises MN9D cells stably transfected with a Nurr1 expressing plasmid under the control of a CMV promoter. A cell line is generated by transfecting cells with a reporter plasmid in which firefly luciferase expression is controlled by multiple copies of a NR4A2-specific DNA binding element; thus agonists of NR4A2 cause an increase in luciferase expression. To identify whether a compound of interest represents an NR4A agonist, compounds of interest were incubated at various concentrations with MN9D cells. After 24 hours of incubation, the cells were processed and assayed for luciferase activity. A similar luciferase reporter assay using CV1 cells is described in reference ii).

It will be appreciated by those skilled in the art that NR4A agonists suitable for use in the medicaments or methods of the invention need not actually bind to a member of the NR4A subgroup, provided that they are still able to activate gene transcription by one or more members of the NR4A subgroup. 6-mercaptopurine (which is a particularly preferred NR4A agonist for use in medicaments or methods of the invention) is an example of an NR4A agonist that is able to activate gene transcription by an NR4A nuclear hormone receptor without binding to the receptor in question.

A therapeutically effective NR4A agonist suitable for use in the medicaments or methods of the invention may be an agonist that is effective to inhibit scarring by at least 10% compared to a suitable control. Preferably a therapeutically effective NR4A agonist may be capable of inhibiting scarring by at least 20%, more preferably at least 50%, even more preferably at least 75% and yet more preferably by at least 90% compared to a suitable control. A most preferred therapeutically effective NR4A agonist may be capable of inhibiting scarring by 100% as compared to a suitable control.

In particular, therapeutically effective NR4A agonists, such as 6-mercaptopurine, suitable for use in the medicaments or methods of the invention may be those able to alter the amount and/or orientation of extracellular matrix components (such as collagen) present in a treated scar and thereby inhibit scarring. A therapeutically effective NR4A agonist suitable for use in the medicaments or methods of the invention may be one that is able to give rise to a treated scar in which the ECM architecture is like that of unwounded tissue.

Preferably a therapeutically effective NR4A agonist, such as 6-mercaptopurine, may be one that is capable of inhibiting scarring at a site to which the agonist is administered. Such a site may be a wound, or scar resulting from the healing of a wound. Alternatively or additionally, such a site may be a site of a fibrotic disorder.

A number of compounds are known that, while not NR4A agonists themselves, are able to undergo conversion to produce an NR4A agonist. In general, it may be preferred that an NR4A agonist suitable for use in the medicaments or methods of the invention may be a compound that is able to act as an NR4A agonist “directly”, that is to say able to agonise NR4A activity when in the form in which it is to be administered to a patient, without the need for conversion or metabolism within a subject in which it is desired to inhibit scarring. The inventors believe that the use of “direct” NR4A agonists in this manner is advantageous for a number of reasons.

The use of direct NR4A agonists in the medicaments or methods of the invention allows a greater degree of control of the amount of an agonist provided to a site of scarring than may be achieved when using indirect agonists. The amount of an NR4A agonist generated by administration of an indirect agonist at a site where scarring is to be inhibited will be determined both by the rate at which the active agonist is generated from the inactive pro-drug, and also be the rate at which the active agonist is metabolically cleared from the site (for example, by conversion to produce an inactive metabolite). As will be appreciated that variable rates of production and metabolism of the indirect NR4A agonist may lead to a situation in which there is no accumulation of a therapeutically effective amount of the NR4A agonist at the site where it is wished to inhibit scarring.

Generally, it may be preferred that NR4A agonists to be used in the medicaments or methods of the invention are agonists having a relatively long half life in the body of a patient to whom the agonist is administered.

It will be appreciated that a mixture of two, or more, different NR4A agonists may be used in the medicaments or methods of the invention to inhibit scarring. Indeed, such use may represent a preferred embodiment of the invention.

### “Therapeutically Effective Amounts”

A therapeutically effective amount of an NR4A agonist, such as 6-mercaptopurine, is any amount of such an NR4A agonist that is able to prevent, reduce or inhibit scarring. Such scarring may be associated with a wound or a fibrotic disorder.

A therapeutically effective amount of an NR4A agonist, such as 6-mercaptopurine, is preferably an amount of such an NR4A agonist which is able to inhibit scarring of a wound (or site at which a wound is to be formed) or a fibrotic disorder (or site at which a fibrotic disorder will occur) to which the NR4A agonist is administered.

A therapeutically effective amount of a medicament of the invention is any amount of a medicament of the invention that is able to inhibit scarring. This inhibition of scarring may preferably be achieved at a site to which the medicament of the invention is administered.

The skilled person will appreciate that an NR4A agonist that has little inherent therapeutic activity will still be therapeutically effective if administered in a quantity that provides a therapeutically effective amount.

A preferred therapeutically effective amount of an NR4A agonist, such as 6-mercaptopurine, to be used in the medicaments or methods of the invention may be an amount that is able to inhibit scarring without inhibiting wound healing. More preferably, a therapeutically effective amount of an NR4A agonist, such as 6-mercaptopurine, to be used in the medicaments or methods of the invention may be an amount that is able to inhibit scarring, and to accelerate wound healing.

A therapeutically effective amount of an NR4A agonist, such as 6-mercaptopurine, to be used in the medicaments or methods of the invention may be an amount that is able to inhibit scarring, but is non-cytotoxic, and so does not impair the function of cells that may contribute to the healing response. A therapeutically effective amount of an NR4A agonist, such as 6-mercaptopurine, to be used in the medicaments or methods of the invention may be an amount that is able to inhibit scarring, but does not impair the function of fibroblasts at the site where scarring is to be inhibited.

A therapeutically effective amount of an NR4A agonist, such as 6-mercaptopurine, or of a medicament of the invention, may preferably be an amount of an agonist or medicament that is effective to inhibit scarring by at least 10% compared to a relevant control. Preferably a therapeutically effective amount of an NR4A agonist, such as 6-mercaptopurine, or of a medicament of the invention, may be capable of inhibiting scarring by at least 20%, more preferably at least 50%, even more preferably at least 75% and yet more preferably of inhibiting scarring by at least 90% compared to a relevant control. A most preferred therapeutically effective amount of an NR4A agonist, such as 6-mercaptopurine, or a medicament of the invention, may be capable of inhibiting scarring by 100% as compared to a relevant control.

The selection of a suitable control will be apparent to one skilled in the art, but by way of guidance, in the event that it is wished to assess inhibition of scarring on healing of wounds treated with an NR4A agonist, such as 6-mercaptopurine, a suitable control may comprise an untreated or control treated wound. In the event that it is wished to assess inhibition of scarring achieved by provision of an NR4A agonist, such as 6-mercaptopurine, to an existing scar, an untreated scar may constitute a suitable control.

Thus a therapeutically effective amount of an NR4A agonist, such as 6-mercaptopurine, or of a medicament of the invention, may be an amount that is effective to reduce scarring occurring on healing of a treated wound by at least 10% compared to scarring occurring on healing of an untreated or control wound. “Treated wounds” and “untreated wounds” or “control wounds” are defined elsewhere in the specification. Preferably a therapeutically effective amount of an NR4A agonist, such as 6-mercaptopurine, or of a medicament of the invention, may be capable of causing a 20% inhibition of scarring, more preferably at least a 50% inhibition, even more preferably at least a 75% inhibition and most preferably at least a 90% inhibition of the scarring occurring on healing of a treated wound as compared to scarring occurring on healing of an untreated or control wound.

In the case of scarring that may otherwise be associated with a fibrotic disorder, a therapeutically effective amount of an NR4A agonist, such as 6-mercaptopurine, or of a medicament of the invention, may be an amount that is effective to reduce scarring of a treated site of fibrosis by at least 10% compared to the amount scarring that would otherwise be present at a comparable untreated site of fibrosis. A “treated site of fibrosis” and “untreated site of fibrosis” are defined further elsewhere in the specification. Preferably a therapeutically effective amount of an NR4A agonist, such as 6-mercaptopurine, or of a medicament of the invention, may be capable of achieving at least a 20% reduction in scarring, more preferably at least 50%, even more preferably at least 75% and most preferably at least a 90% reduction in scarring compared to scarring present at a comparable untreated site of fibrosis.

Suitable experimental or clinical models for the assessment of scarring (and thus of inhibition of scarring) will be well known to those skilled in the art. Suitable examples are set out elsewhere in the present specification.

Suitable experimental models of scarring, whether associated with healing of a wound or with a fibrotic disorder, and suitable controls that may be used when assessing scarring occurring in such models, are considered elsewhere in the specification. A preferred model is described in detail in the Experimental Results section.

Quantitative assessment of the extent of scarring (allowing generation of percentage values indicative of inhibition of scarring, as referred to throughout the specification) may be undertaken using any suitable protocol. It is preferred that such quantitative values are recorded using a suitable visual analogue scale (VAS). Suitable VAS may be used to assess scarring macroscopically or microscopically. Suitable criteria that may be considered in either macroscopic or microscopic assessment of scars are set out elsewhere in the specification. It may be preferred that assessment of scarring considers the microscopic structure of a scar.

A therapeutically effective amount of an NR4A agonist, such as 6-mercaptopurine, may preferably be an amount able to therapeutically alter the abundance and/or orientation of ECM components (such as collagen) in a treated scar.

A medicament of the invention will be capable of providing a therapeutically effective amount of an NR4A agonist, such as 6-mercaptopurine, when administered in a suitable amount and via a suitable route. Preferably a medicament of the invention may be provided in the form of one or more dosage units. Each dosage unit may comprise a therapeutically effective amount of an NR4A agonist, such as 6-mercaptopurine, or a known fraction or multiple of such a therapeutically effective amount.

As noted above, preferred therapeutically effective amounts of an NR4A agonist, such as 6-mercaptopurine, for use in the medicaments or methods of the invention may be capable of accelerating the healing of a treated wound. The acceleration of healing of wounds within the context of the present invention may be understood to encompass any increase in the rate of healing of a treated wound as compared to the rate of healing occurring in a control-treated or untreated wound. The rate of healing of wounds attained in accordance with the invention may readily be compared with that taking place in control-treated or untreated wounds using any suitable model of wound healing known in the art. Suitable models in which the rate of wound healing may be assessed are set out elsewhere in the specification.

Accelerated healing of a wound achieved using the medicaments or methods of the invention may preferably lead to a treated wound healing at a rate at least 5% faster than a control wound or untreated wound, preferably at a rate at least 10% faster, more preferably at least 15%, 20% or 25% faster; yet more preferably at least 50% faster, still more preferably at least 75% faster, and most preferably 100% (or more) faster. Suitable methods by which acceleration of the healing of wounds may be quantified to assess improvements in the rate of healing are described elsewhere in the specification.

The inventors have found that NR4A agonists, such as 6-mercaptopurine, are able to inhibit scarring, and to accelerate wound healing, across a broad range of doses, encompassing all doses investigated thus far.

The inventors believe that the provision of between approximately 0.1 ng and 1500 ng of an NR4A agonist, such as 6-mercaptopurine, per centimetre of wound or fibrosis (in a single incidence of treatment) may constitute a therapeutically effective amount in accordance with the present invention. Preferably a therapeutically effective amount of an NR4A agonist, such as 6-mercaptopurine, may be between about 1 ng and 1000 ng per centimetre of wound or fibrosis in a single incidence of treatment.

The inventors believe that an NR4A agonist, such as 6-mercaptopurine, may be provided in a therapeutically effective amount of between approximately 0.59 pmol and 8.85 nmol of the agonist per centimetre of wound or fibrosis (in a single incidence of treatment). Preferably a therapeutically effective amount of an NR4A agonist, such as 6-mercaptopurine, may be between about 5.9 pmol and 5.9 nmol per centimetre of wound or fibrosis in a single incidence of treatment.

It may be preferred that a therapeutically effective amount of an NR4A agonist, such as 6-mercaptopurine, considered in the preceding paragraphs be administered twice to a wound or site of fibrosis over a period of approximately 24 hours. The inventors believe that these therapeutically effective amounts (i.e. between approximately 1.18 pmol and 17.7 nmol, and preferably between about 11.8 pmol and 11.8 nmol) may also constitute preferred therapeutically effective amounts to be administered over the course of an entire regime of treatment.

Preferred therapeutically effective amounts of an NR4A agonist (either generally or with reference to specific selected agonists, such as 6-mercaptopurine) may be investigated using in vitro and in vivo models, and suitable assessments of efficacy made with reference to various parameters for the measurement of scarring, as described elsewhere in the specification.

The skilled person will recognise that the information provided in the preceding paragraphs as to amounts of NR4A agonists, such as 6-mercaptopurine, which may be administered to wounds or sites of fibrotic disorders in order to inhibit scarring, may be varied by the skilled practitioner in response to the specific clinical requirements of an individual patient. A physician (such as a physician with responsibility for the treatment of the patient in question) may determine suitable variations empirically, with reference to a range of factors including (but not limited to) the nature of the tissue to be treated, the area and/or depth of the wound or fibrosis to be treated, the severity of the wound or fibrosis, and the presence or absence of factors (such as infection) that may complicate healing or increase the likely magnitude of scarring, the nature of the scarring to be inhibited, any level of accelerated wound healing that is to be attained, and with reference to any inhibition of scarring already achieved.

In the event that an NR4A agonist, such as 6-mercaptopurine, is to be administered via topical administration, the amount provided may be altered depending on permeability of the tissue or organ to which the topical composition is administered. Thus, in the case of relatively impermeable tissues or organs, it may be preferred to increase the amount of an NR4A agonist, such as 6-mercaptopurine, to be administered. Such an increased amount of an NR4A agonist, such as 6-mercaptopurine, may still represent a therapeutically effective amount, if the amount of the agent taken up into the tissue or organ where scarring is to be inhibited is therapeutically effective.

It will be appreciated that the guidance provided herein, as to doses and amounts of an active agent to be used, is applicable both to medicaments of the invention, and also to the methods of the invention.

The inventors have found that in one particularly preferred embodiment 6-mercaptopurine may be administered in the form of a 1 ng/100 μl injectable solution, with 100 μl of such a solution provided per centimetre of wound or fibrosis over an approximately 24 hour period. The solution may preferably be provided by intradermal injection, and may be provided for two, or more, days.

In another particularly preferred embodiment 6-mercaptopurine may be administered in the form of a 1000 ng/100 μl injectable solution, with 100 μl of such a solution provided per centimetre of wound or fibrosis over an approximately 24 hour period. The solution may preferably be provided by intradermal injection, and may be provided for two, or more, days.

In the case where the paragraphs above consider the administration of a specified amount of a medicament per linear cm of a wound it will be appreciated that this volume may be administered to either one or both of the margins of a wound to be treated (i.e. in the case of a reference to 100 μl of a medicament, this may be administered as 100 μl to either of the wound margins, or as 50 μl to each of the wound margins to be joined together).

### Centimetre of Wound or Fibrosis

In the context of the present disclosure, a “centimetre of wound” or a “centimetre of fibrosis” represents a unit by which the size of site at which scarring is to be prevented, reduced or inhibited may be measured. For the present purposes, a centimetre of wound may be taken to encompass a site where a wound is to be formed, as well as a wounded site, or both margins of a wounded site (should such margins exist).

A centimetre of wound may be taken to comprise any square centimetre of a body surface that is wounded in whole or in part. For example, a wound of two centimetres length and one centimetre width (i.e. with a total surface area of two square centimetres) will be considered to constitute “two wound centimetres”, while a wound having a length of two centimetres and a width of two centimetres (i.e. a total surface area of four square centimetres) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

A centimetre of fibrosis should be construed in a similar manner, i.e. to encompass any square centimetre of the body in which scarring has occurred (either as a result of a fibrotic disorder, or the healing of a wound), as well as any square centimetre in which scarring may be expected to occur as a result of a fibrotic disorder.

The size of a site in wound centimetres, or centimetres of fibrosis, should generally be assessed when the wound is in its relaxed state (i.e. when the body site bearing the site to be measured is in the position adopted when the body is at rest). In the case of the skin, the relevant size should be assessed when the skin is not subject to external tension.

### “Medicaments of the Invention”

For the purposes of the present disclosure, medicaments of the invention should be taken as encompassing any medicament manufactured in accordance with any aspect or embodiment of the invention.

Medicaments of the invention will generally comprise a pharmaceutically acceptable excipient, diluent or carrier in addition to the NR4A agonist, such as 6-mercaptopurine.

Medicaments of the invention may preferably be in the form of an injectable solution comprising an NR4A agonist, such as 6-mercaptopurine. Solutions suitable for localised injection (and in particular for intradermal injection) constitute particularly preferred forms of the medicaments of the invention.

### Preferred Body Sites

The inventors believe that the prevention, reduction or inhibition of scarring using an NR4A agonist, such as 6-mercaptopurine, can be effected at any body site other than the eye, and in any non-ocular tissue or organ. The skin represents a preferred site at which scarring may be prevented, reduced or inhibited utilising the medicaments or methods of the invention. Without wishing to limit the scope of the invention, the following passages provide guidance as to specific tissues and body sites that may benefit from inhibition of scarring using the medicaments or methods of the invention.

The use of an NR4A agonist, such as 6-mercaptopurine, to inhibit scarring may bring about a notable improvement in the cosmetic appearance of an injured area thus treated. Cosmetic considerations are important in a number of clinical contexts, particularly when scars may be formed at prominent body sites such as the face, neck and hands.

Consequently it is a preferred embodiment that the medicaments and methods of the invention be used to inhibit scarring at sites where it is desired to improve the cosmetic appearance of a scar formed.

In addition to its cosmetic impact, scarring of the skin is responsible for a number of deleterious effects afflicting those suffering from such scarring. For example, scarring of the skin may be associated with reduction of physical and mechanical function, particularly in the case of contractile scars and/or situations in which scars are formed across joints (articulations). The contraction exhibited by contractile scars of this kind is more pronounced than wound contraction that occurs as a normal part of the healing process, and may be distinguished from such normally occurring contraction in that it continues long after the healing process has ended (i.e. after wound closure). In cases of scars located in the area of joints the altered mechanical properties of scarred skin, as opposed to unscarred skin, and the effects of scar contraction may lead to dramatically restricted movement of a joint so affected. Accordingly, it is a preferred embodiment that suitable medicaments and methods of the invention be used to inhibit scarring covering joints of the body (whether such scars result from the healing of wounds covering the joint, or are associated with fibrotic disorders covering the joint). In another preferred embodiment suitable medicaments and methods of the invention may be used to inhibit scarring at increased risk of forming a contractile scar (in the case of scarring that results from the healing of wounds this may include wounds of children, and/or wounds produced by burns).

The extent of scar formation, and hence extent of cosmetic or other impairment that may be caused by the scar, may also be influenced by factors such as the tension of the site at which the scar is formed (and in the case of scarring that results from the healing of a wound, the tension at the site where the wound is formed). For example, it is known that skin under relatively high tension (such as that extending over the chest, or associated with lines of tension) may be prone to formation of more severe scars than at other body sites. Thus in a preferred embodiment suitable medicaments and methods of the invention may be used to inhibit scarring at sites of high skin tension. The medicaments and methods of the invention may, for example, be used to inhibit scarring that results from the healing of wounds located at sites of high skin tension.

It will be appreciated that tissues other than the skin may also be subject to scarring, whether associated with wounds or fibrotic disorders. The medicaments and methods of the invention may also be of benefit in inhibiting scarring associated with wounds or fibrotic disorders in these tissues.

The healing of wounds involving the peritoneum (the epithelial covering of the internal organs, and/or the interior of the body cavity) may frequently give rise to adhesions. Such adhesions are formed by bands of fibrous scar tissue, and can connect the loops of the intestines to each other, or the intestines to other abdominal organs, or the intestines to the abdominal wall. Adhesions can pull sections of the intestines out of place and may block passage of food. Adhesions are also a common sequitur of surgery involving gynaecological tissues. Incidences of adhesion formation may be increased in wounds that are subject to infection (such as bacterial infection) or exposure to radiation.

Scarring in the central and peripheral nervous system may be inhibited using an NR4A agonist, such as 6-mercaptopurine. Such scarring may arise as a result of surgery or trauma. Inhibition of scarring in the nervous system may be assessed by assays of nerve function (e.g. sensory or motor tests), where inhibition of scarring is indicated by improved performance in such tests.

Scarring in the blood vessels, e.g. following anastomotic surgery, can lead to myointimal hyperplasia and reduction in the volume of the blood vessel lumen (restenosis). A therapeutically effective amount of an NR4A agonist, such as 6-mercaptopurine, may be provided to blood vessels by any suitable means in order to inhibit such scarring.

The medicaments or methods of the invention may be used to inhibit scarring in tendons and ligaments. Such scarring may otherwise be expected to occur following surgery or trauma involving tissues of this type.

Agents of the invention may be used to inhibit scarring in a range of “internal” wounds or fibrotic disorders (i.e. wounds or fibrotic disorders occurring within the body, rather than on an external surface). Examples of internal wounds include penetrative wounds that pass through the skin into underlying tissues, and wounds associated with surgical procedures conducted within the body.

### Preferred Wounds

The inventors believe that medicaments or methods utilising NR4A agonists, such as 6-mercaptopurine, may be used to beneficially inhibit scarring in all types of wounds.

Examples of specific wounds in which scarring may be inhibited using the medicaments and methods of the invention include, but are not limited to, those independently selected from the group consisting of: wounds of the skin; wounds subject to capsular contraction (which is common surrounding breast implants); wounds of blood vessels; wounds of the central and peripheral nervous system (where prevention, reduction or inhibition of scarring may enhance neuronal reconnection and/or neuronal function); wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce or inhibit scarring associated with wounds of the skin.

The inventors believe that the ability of the medicaments and methods of the invention to inhibit scarring may reduce the occurrence of adhesions (such as those occurring in the abdomen, pelvis, thorax or spine). Accordingly, the use of an NR4A agonist, such as 6-mercaptopurine, to prevent the formation of adhesions represents a preferred embodiment of the invention. The use of medicaments or methods of the invention in the inhibition of scarring involving the peritoneum is another preferred embodiment.

The medicaments and methods of the invention may be useful in the inhibition of scarring that may occur on healing of infected wounds or wounds exposed to radiation. Wounds of this sort may be at increased risk of developing into chronic wounds, and so may derive particular benefit from the medicaments and methods of the invention that are able to accelerate wound healing.

Incisional wounds constitute preferred wounds scarring resulting from which may be inhibited by the medicaments and methods of the invention. Surgical incisional wounds may constitute a particularly preferred group of wounds in respect of which scarring may be inhibited utilising the medicaments and methods of the invention.

It is a preferred embodiment that the medicaments and methods of the invention be used to inhibit scarring associated with cosmetic surgery. Since the great majority of cosmetic surgeries consist of elective surgical procedures it is readily possible to administer an NR4A agonist, such as 6-mercaptopurine, prior to surgery, and/or immediately following closure of the wound (e.g. with sutures), and this use represents a particularly preferred embodiment of the invention. In the case of elective surgical procedures a preferred route by which an NR4A agonist, such as 6-mercaptopurine, may be administered is via intradermal injection. Such injections may form raised blebs, which may then be incised as part of the surgical procedure, or alternatively the bleb may be raised by injecting the wound margins after the wound has been closed e.g. by sutures.

The cosmetic outcome of surgical procedures is also an important consideration in plastic surgery, and the use of methods or medicaments of the invention to inhibit scarring associated with plastic surgery constitutes a further preferred embodiment of the invention.

Scar revisions are surgical procedures in which existing scars are “revised” (for example through excision or realignment) in order to reduce the cosmetic and/or mechanical disruption caused by the existing scar. Scar revision may involve the removal of an existing scar so that a less noticeable, or otherwise deleterious, scar may be produced in its place. The use of the medicaments or methods of the invention in procedures associated with scar revision represents a preferred use in accordance with the present invention.

There are many surgical procedures that may be used in scar revision to allow realignment of wounds and scars such that they are subject to reduced tension. Probably the best known of these is “Z-plasty” in which two V-shaped flaps of skin are transposed to allow rotation of a line of tension. In a more preferred embodiment the medicaments and methods of the invention may be used to inhibit scarring of wounds during surgical revision of disfiguring scars, such as by Z-plasty.

It may generally be preferred that the medicaments or methods of the invention, in which an NR4A agonist, such as 6-mercaptopurine, is used to inhibit scarring, be used to inhibit non-pathological scarring (e.g. scarring occurring in a patient without a history of, or susceptibility to, keloids, hypertrophic scars, or the like). A history of pathological scarring, or susceptibility to pathological scarring, may be identified with reference to a patient's clinical history, or by means of tests for genetic markers known to be associated with a predisposition to pathological scarring.

It is often clinically necessary to surgically revise hypertrophic scars or keloids, the aim of such revision being to replace the relatively severe pathological scar with a less noticeable non-pathological scar. Revision in this manner may be designed to reduce incidence of pathological scarring by, for example, reducing tension at the site of the revised scar, or by taking other appropriate steps to inhibit scarring. Thus, it is a further preferred embodiment of the invention that the medicaments or methods herein described be used to inhibit non-pathological scarring that results from wounds produced during surgical revision of pathological scars.

It is recognised that wounds resulting from burns injuries (which for the purposes of the present invention may be taken to encompass exposure to heated gasses or solids, as well as scalding injuries involving hot liquids; “freezer burn” injuries caused by exposure to extreme low temperatures; radiation burns; and chemical burns, such as those caused by caustic agents) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Furthermore, burns injuries, and particularly those suffered by children, have an elevated risk of producing contractile scars, such as hypertrophic scars. These may increase both the cosmetic and mechanical impairment associated with scarring after burns. Accordingly, it is a preferred embodiment that medicaments and methods of the invention be used to inhibit scarring resulting from burns injuries.

The ability of an NR4A agonist, such as 6-mercaptopurine, to inhibit scarring is of great utility in the inhibition of scarring associated with grafting procedures. In particular, the medicaments and methods of the invention may be used to inhibit scarring that results from wounds associated with grafting procedures. Inhibition of scarring using the medicaments and methods of the invention is of benefit both at a graft donor sites and graft recipient sites. The scar inhibitory effects of the medicaments and methods of the invention are able to inhibit scarring that may otherwise occur at sites where tissue for grafting is removed, or that may be associated with the healing and integration of grafted tissue. The inventors believe that the methods and medicaments of the invention confer advantages in the inhibition of scarring that may otherwise be associated with grafts utilising skin, artificial skin, or skin substitutes.

The inventors also believe that the medicaments and methods of the invention may be used to inhibit scarring associated with encapsulation. Encapsulation is a form of scarring that occurs around sites at which implant materials (such as biomaterials) have been introduced into the body. Encapsulation is a frequent complication associated with breast implants, and the use of an NR4A agonist, such as 6-mercaptopurine, to inhibit encapsulation in this context is a preferred embodiment of the invention.

The medicaments and methods of the invention may be used to inhibit scarring that results from healing of wounds selected from the group consisting of: abrasions (also commonly referred to as “scrapes”, these are shallow injuries which frequently cover a relatively large area); avulsions (when an entire bodily structure, or a part of such a structure, is forcibly pulled away from its site); crush wounds; incisional wounds; lacerations; punctures; and missile wounds. All of these different types of wounds may be suffered by the skin, among other tissues or organs, and all may, to a greater or lesser extent, result in scarring.

The wounds resulting from surgical procedures are most commonly incisional wounds, and these are a frequent cause of scarring. Accordingly it is a preferred embodiment that the medicaments and methods of the invention be used in the inhibition of scarring resulting from incisional wounds, such as surgical wounds. It is believed that each year 84 million surgical procedures are conducted worldwide in which the skin is incised. Accordingly, it can be seen that the potential market for, potential benefits provided by, the medicaments and methods of the invention are very large indeed.

The inventors believe that the medicaments or methods of the invention may be of use in inhibiting scarring associated with full thickness or partial thickness wounds (respectively wounds in which the epithelial layer is either totally or partly compromised). Preferred examples of partial thickness wounds, scarring associated with which may be inhibited using the medicaments or methods of the invention, include “skin peels” such as “chemical peels” (such as alphahydroxy acid peels, trichloroacetic acid peels or phenol peels) or “laser peels”; wounds associated with dermabrasion; and wounds associated with dermaplaning. It may particularly be preferred that the medicaments or methods of the invention be used to inhibit scarring associated with partial thickness wounds occurring at cosmetically important sites (such as the face), which may frequently be the subject of skin peel treatment.

### Preferred Fibrotic Disorders

The medicaments or methods of the invention utilising NR4A agonists, such as 6-mercaptopurine, may be used to prevent, reduce or inhibit scarring associated with any fibrotic disorder. By way of example, and without limiting the scope of protection sought, the medicaments or methods of the invention may preferably be used to treat fibrotic disorders independently selected from the group consisting of: skin fibrosis; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; chronic obstructive pulmonary disease; fibrosis following myocardial infarction; central nervous system fibrosis, such as fibrosis following stroke; fibrosis associated with neuro-degenerative disorders such multiple sclerosis; restenosis; endometriosis; ischemic disease and radiation fibrosis.

### Prevention, Reduction or Inhibition of Scarring

The prevention, reduction or inhibition of scarring within the context of the present invention should be understood to encompass any degree of prevention, reduction or inhibition in scarring achieved on healing of a treated wound, or in a treated scar or treated site of a fibrotic disorder as compared to the level of scarring occurring on healing of a control-treated or untreated wound, or in an untreated scar, or at an untreated site of a fibrotic disorder. Throughout the specification references to “prevention”, “reduction” or “inhibition” of scarring are generally to be taken, except where the context requires otherwise, to represent substantially equivalent activities, involving equivalent mechanisms mediated by NR4A agonists, such as 6-mercaptopurine.

For the sake of brevity, the present specification will primarily refer to “inhibition” of scarring utilising an NR4A agonist, such as 6-mercaptopurine. However, such references should be taken, except where the context requires otherwise, to also encompass the prevention or reduction of scarring using such compounds. Similarly, references to “prevention” of scarring using an NR4A agonist, such as 6-mercaptopurine, should, except where the context requires otherwise, be taken also to encompass the treatment of scarring using such compounds.

The inhibition of scarring achieved using methods and medicaments of the invention may be assessed and/or measured with reference to the microscopic and/or macroscopic appearance of a treated scar Inhibition of scarring may also suitably be assessed by comparing the microscopic and/or macroscopic appearance of a treated scar with the microscopic and/or macroscopic appearance of an untreated scar. In general, it may be preferred that inhibition of scarring using medicaments or methods utilising an NR4A agonist, such as 6-mercaptopurine, are assessed with reference to the microscopic appearance of a treated scar.

Suitable methods and parameters by which scarring may be assessed in treated scars or control scars (and hence any inhibition of scarring identified) are described elsewhere in the specification, as are methods by which such assessments may be captured and quantified (if so required).

In the event that scarring is assessed quantitatively, it may be preferred that inhibition of scarring be indicated by a statistically significant decrease in a treated wound, scar or site of fibrosis, as compared to a control counterpart.

### “Treated Wounds”, “Untreated Wounds”, “Treated Sites of Fibrosis”, “Untreated Sites of Fibrosis”, “Treated Scars” and “Untreated Scars”

Treatment of wounds with a therapeutically effective amount of an NR4A agonist, such as 6-mercaptopurine, will inhibit the scarring that may otherwise be expected to occur on the healing of untreated wounds.

For present purposes an “untreated wound” should be considered to be any wound that has not been exposed to a therapeutically effective amount of an NR4A agonist, such as 6-mercaptopurine. A “diluent control-treated wound” will be an untreated wound to which a control diluent has been administered, and a “naïve control” will be an untreated wound made without administration of an NR4A agonist, such as 6-mercaptopurine, and without a suitable control diluent, and left to heal without therapeutic intervention.

In contrast, a “treated wound” may be considered to be a wound exposed to a therapeutically effective amount of an NR4A agonist, such as 6-mercaptopurine. Thus a treated wound may be a wound which has been provided with a medicament of the invention, or which has received treatment in accordance with the methods of the invention.

For the present purposes a “treated scar” should be taken to encompass:
i) a scar that results from healing of a treated wound (i.e. a wound treated with a therapeutically effective amount of an NR4A agonist, such as 6-mercaptopurine); and/or
ii) a scar produced at a site of a fibrotic disorder that has been treated with a therapeutically effective amount of an NR4A agonist, such as 6-mercaptopurine; and/or
iii) a scar to which a therapeutically effective amount of an NR4A agonist, such as 6-mercaptopurine, has been administered.

By way of contrast, an “untreated scar” should be taken to encompass:
i) a scar that results from healing of an untreated wound (for example a wound treated with a placebo, control, or standard care); and/or
ii) a scar to which a therapeutically effective amount of an NR4A agonist, such as 6-mercaptopurine, has not been administered.

Untreated scars may typically be used as comparators in assessing the inhibition of scarring that may be evident in a treated scar. Suitable comparator untreated scars of this type may preferably be matched to the treated scar with reference to one or more criteria independently selected from the group consisting of: scar age; scar size; scar site; Body Mass Index of patients; patient age; patient race and patient gender.

Treatment of a site of a fibrotic disorder with a therapeutically effective amount of an NR4A agonist, such as 6-mercaptopurine, is able to inhibit scarring, and will give rise to a “treated site of fibrosis”, which will comprise a treated scar. The scarring at such a treated site of fibrosis may be compared with that occurring in an untreated or control site of a fibrotic disorder (i.e. a site which has not been provided with a therapeutically effective amount of an NR4A agonist, such as 6-mercaptopurine).

The inventors believe that treatment of fibrotic disorders in this manner may have an impact on the macroscopic and/or microscopic appearance of the scar associated with the fibrotic disorder, such that the macroscopic and/or microscopic structure of a scar at a treated site of fibrosis will be more akin to that found in normal non-fibrotic tissue. For example, in the case of fibrosis involving the skin, a treated scar may, when viewed microscopically, exhibit an abundance and orientation of ECM molecules, such as collagen, that is more similar to that found in normal skin than that found in untreated scars.

### Models of Scarring

In the case of inhibition of scarring that results from the healing of a wound, a suitable animal model in which the therapeutic effectiveness of an NR4A agonist, such as 6-mercaptopurine, may be assessed, and in which a therapeutically effective amount of an active agent may be determined, may involve providing the an NR4A agonist, such as 6-mercaptopurine, to incisional or excisional wounds of experimental subjects (either humans subjects, or non-human animals such as mice, rats or pigs), and assessing the scarring that results on healing of the wound. Suitable models may utilise full thickness or partial thickness wounds depending on the wounding that it is intended to treat. Examples of models of full or partial thickness wound healing are well known to those skilled in the art.

In the case of inhibition of scarring associated with fibrotic disorders, the commonality of the biological mechanisms underlying scarring means that this scarring may also be investigated using incisional or excisional wound healing models of the type outlined above.

However, the skilled person will also be aware of specific experimental models of fibrotic disorders that may be used to further investigate the therapeutic effectiveness of an NR4A agonist, such as 6-mercaptopurine, in this context. For example, administration of CCl₄ to experimental animals allows the generation of an experimental model of fibrosis of the liver that may be used to assess effectiveness of an NR4A agonist, such as 6-mercaptopurine, in the context of inhibiting scarring associated with liver fibrosis. Furthermore, an experimental model of glomerulonephritis may be established either by injection of suitable serum proteins into an experimental animal or injection of nephrotoxic serum, and either of these animal models may be useful in assessment of an NR4A agonist, such as 6-mercaptopurine, in the inhibition of scarring associated with kidney fibrosis.

The experimental models described above may also allow identification of particular effective routes or regimes by which an NR4A agonist, such as 6-mercaptopurine, may be administered. These routes or regimes may provide notable advantages in the context of the medicaments and methods of the present invention, and these may give rise to further aspects of the invention.

### Assessment of Scarring, and of Inhibition of Scarring

The extent of inhibition of scarring that may be required in order to achieve a therapeutic effect will be apparent to, and may readily be determined by, a clinician responsible for the care of the patient. The clinician may undertake a suitable determination of the extent of inhibition of scarring that has been achieved using an NR4A agonist, such as 6-mercaptopurine, in order to assess whether or not a therapeutic effect has been achieved, or is being achieved. Such an assessment may, but need not necessarily, be made with reference to suggested methods of measurement described herein.

The extent to which inhibition of scarring utilising an NR4A agonist, such as 6-mercaptopurine, is achieved may be assessed with reference to the effects that such an active agent may achieve in human patients treated with the methods or medicaments of the invention. Alternatively, inhibition of scarring that may be achieved by an NR4A agonist, such as 6-mercaptopurine, may be assessed with reference to experimental investigations using suitable in vitro or in vivo models. The use of experimental models to investigate inhibition of scarring may be particularly preferred in assessing the therapeutic effectiveness of particular NR4A agonists, or in establishing therapeutically effective amounts of such agonists, such as 6-mercaptopurine.

Animal models of scarring represent preferred experimental models for in vivo assessment of the extent of scar inhibition that may be achieved using the medicaments or methods of the invention. Suitable models may be used specifically to investigate scarring that results from the healing of a wound, and, additionally or alternatively, to investigate scarring associated with fibrotic disorders. Suitable models of both types will be known to those skilled in the art, and examples of such models are described elsewhere in the specification for illustrative purposes.

A number of methods for the assessment of scarring have been developed, primarily with regard to scarring of the skin (being the body's largest organ, and the organ in which scars have the greatest cosmetic impact). Accordingly, the following description of methods for assessing the scar inhibitory activity of medicaments and methods of the invention will primarily be described with reference to assessment of scarring in the skin. However, the skilled person will immediately appreciate that many of the factors that are relevant when assessing scarring in the skin are also relevant to assessment of scarring in other organs or tissues. Accordingly the skilled person will recognise that, except for where the context requires otherwise, the parameters suggested below, in the context of assessment of scars of the skin, may also be applicable to assessment of scarring in tissues other than the skin.

Assessment of scarring may take into consideration the macroscopic appearance of scars and/or the microscopic appearance of scars. It may be preferred that assessment of scarring be conducted with reference to the microscopic appearance of scar. Since the microscopic appearance of a scar reflects the internal structure of the scar, this may provide a valuable indication as to the mechanical and physical properties of the scar. Preferably an assessment of scarring may be an assessment providing a quantifiable value indicative of the degree of scarring present.

The inhibition of scarring achieved using methods and medicaments of the invention may be assessed and/or measured with reference to the microscopic and/or macroscopic appearance of a treated scar. Inhibition of scarring may also suitably be assessed with reference to microscopic and/or macroscopic appearance of a treated scar as compared to the appearance of an untreated scar. Inhibition of scarring will be demonstrated when the appearance of a treated scar is assessed as more similar to unwounded tissue than is the appearance of an untreated or control treated scar.

In considering the macroscopic appearance of a scar resulting from a treated wound, the extent of scarring, and hence the magnitude of any inhibition of scarring achieved, may be assessed with reference to a number of parameters. Suitable parameters may be considered individually or in combination.

The extent of scarring, and so any inhibition of scarring achieved, may be assessed by macroscopic clinical assessment of scars. This may be achieved by the direct assessment of scars upon a subject; or by the assessment of photographic images of scars; or of silicone moulds taken from scars, or positive plaster casts made from such moulds. Macroscopic characteristics of a scar which may be considered when assessing scarring include:
i) Colour of the scar. Scars may typically be hypopigmented or hyperpigmented with regard to the surrounding skin Inhibition of scarring may be demonstrated when the pigmentation of a treated scar more closely approximates that of unscarred skin than does the pigmentation of an untreated scar. Scars may often be redder than the surrounding skin. In this case inhibition of scarring may be demonstrated when the redness of a treated scar fades earlier, or more completely, or to resemble more closely the appearance of the surrounding skin, compared to an untreated scar. Colour can readily be measured, for example by use of a spectrophotometer.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on the patient (e.g. by means of profilometry), or indirectly, (e.g. by profilometry of moulds taken from a scar).
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can also be measured either directly on the patient (e.g. by means of profilometry), or indirectly (e.g. by profilometry of moulds taken from a scar).
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two of the parameters, even more preferably at least three of the parameters, and most preferably at least four of these parameters (for example, all four of the parameters set out above).

One preferred method for the macroscopic assessment of scars is holistic assessment. This may be accomplished by means of assessment of macroscopic photographs by an expert panel or a lay panel, or clinically by means of a macroscopic assessment by a clinician or by patients themselves. Assessments may be captured by means of a VAS (visual analogue scale) or a categorical scale. Examples of suitable parameters for the assessment of scarring (and thereby of any reduction of scarring attained) are described below. Further examples of suitable parameters, and means by which assessment of such parameters may be captured, are described by Duncan et al. (2006), Beausang et al. (1998) and van Zuijlen et al. (2002).

Microscopic assessment of scars will generally employ histological analysis of the microscopic structure of scars. Suitable parameters for the microscopic assessment of scars may include:
i) Thickness of extracellular matrix (ECM) fibres. Scars typically contain thinner ECM fibres than are found in unscarred skin Inhibition of scarring may be demonstrated when the thickness of ECM fibres in a treated scar more closely approximates the thickness of ECM fibres found in unscarred skin than does the thickness of fibres found in an untreated scar.
ii) Orientation of ECM fibres. ECM fibres found in scars tend to exhibit a greater degree of alignment with one another than do those found in unscarred skin (which have a random “basket weave” orientation). Accordingly, inhibition of scarring may be demonstrated when the orientation of ECM fibres in a treated scar more closely approximates the orientation of ECM fibres found in unscarred skin than does the orientation of such fibres found in an untreated scar.
iii) Abundance of ECM components. Scars typically contain an increased amount of ECM components such as collagen when compared to unscarred skin. An inhibition of scarring may be indicated when a treated scar contains reduced abundance of ECM components when compared to untreated or control treated scars, or when a treated scar contains an abundance of ECM components that is more similar to unscarred skin than the abundance contained in an untreated or control scar.
iv) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar.
v) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar.

One or more of the parameters suggested above may be used to form the basis of a visual analogue scale for microscopic assessment of scarring. Inhibition of scarring may be indicated when the quality of a treated scar is closer to that of unscarred skin than is the quality of an untreated or control scar.

It is surprising to note that the overall appearance of scars, such as those of the skin, is little influenced by the epithelial covering of the scar, even though this is the part of the scar that may be most readily seen by the observer. Instead, the inventors find that the properties of the connective tissue (such as that making up the dermis, or neo-dermis) present within the scar have greater impact on the perception of extent of scarring, as well as on the function of the scarred tissue. Accordingly assessments of criteria associated with the connective tissues such as the dermis, rather than epithelia such as the epidermis, may prove to be the most useful in assessing inhibition of scarring. The thickness of ECM fibres and orientation of ECM fibres within the connective tissue may be favoured parameters, for assessing inhibition of scarring. It may be desirable to assess ECM organisation and abundance in the papillary dermis and the reticular dermis separately when considering scar quality.

A treated scar may preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, and most preferably all four of these parameters. Macroscopic and microscopic parameters may be combined in assessing inhibition of scarring (i.e. assessing at least one parameter used in macroscopic assessment and at least one parameter used in microscopic assessment).

There are a number of ways in which assessments of scarring may be captured and quantified. Suitable methods may be used to capture macroscopic or microscopic assessments of scarring, and may generally be performed either directly (on the patient), or indirectly (on photographs or moulds taken from the patient). Without limitation, examples of means by which assessment of scarring may be captured include:

### Assessment Using Visual Analogue Scale (VAS) Scar Scores.

Assessments of scars may be captured using a scarring-based VAS. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar. Use of a VAS in this manner allows for easy capture and quantification of assessment of scarring. VAS scoring may be used for the macroscopic and/or microscopic assessment of scarring.

Merely by way of example, a suitable macroscopic assessment of scarring may be carried out using a VAS consisting of a 0-10 cm line representing a scale, from left to right, of 0 (corresponding to normal skin) to 10 (indicative of a bad scar). A mark may be made by an assessor on the 10 cm line based on an overall assessment of the scar. This may take into account parameters such as the height, width, contour and colour of the scar. The best scars (typically of small width, and having colour, height and contour like normal skin) may be scored towards the “normal skin” end of the scale (the left hand side of the VAS line) and bad scars (typically large width, raised profile and with uneven contours and whiter colour) may be scored towards the “bad scar” end of the scale (the right hand side of the VAS line). The marks may then be measured from the left hand side to provide the final value for the scar assessment in centimetres (to 1 decimal place).

For microscopic assessment, the scars may be excised from experimental subjects (preferably incorporating a small amount of surrounding normal tissue) and fixed (for example in 10% buffered formal saline). The fixed tissue may then be processed for wax histology. Histological slides may be stained using a suitable protocol to allow assessment of scarring (such as Masson's trichrome or Mallory's trichrome), and scarring assessed by a assessor using a microscopic VAS. A suitable VAS may consist of a 0-10 cm line representing a scale, from left to right, of 0 (corresponding to normal skin) to 10 (indicative of a bad scar). A mark may be made on the 10 cm line based on an overall assessment of the scar taking into account parameters such as collagen fibre spacing, orientation and thickness. The best scars (typically narrow scars with thick and randomly organised collagen fibres that have normal spacing between fibres, similar to that found in unscarred dermis) will be scored towards the “normal skin” end of the scale (the left hand side of the VAS line) and bad scars (typically wide scars with thin densely packed parallel collagen fibres) will be scored towards the “bad scar” end of the scale (the right hand side of the VAS line). The marks can then be measured from the left hand side to provide the final value for the scar assessment in centimetres (to 1 decimal place).

Assessment with Regard to an Overall Categorical Scale.

Assessment of scarring may be captured by allocating scars to different categories based on either textual descriptions of the scar assessed (e.g. “barely noticeable”, “blends well with normal skin”, “distinct from normal skin”, etc.), or by comparing a treated scar and an untreated or control scar, noting any differences between these, and allocating the differences to selected categories (e.g. “mild difference”, “moderate difference”, “major difference”, etc.). These assessments may be conducted with reference to the over-all appearance of the scar(s) assessed. Inhibition of scarring may be demonstrated when an assessment indicates that treated scars are allocated to at least one more favourable category than are untreated or control scars. Assessment of this sort may be performed by the patient, by an investigator, by an independent panel, or by a clinician.

### Assessment of Scar Height, Scar Width, Scar Perimeter, Scar Area or Scar Volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, or using plaster casts of impressions of the scar. The skilled person will also be aware of further non-invasive methods and devices that can be used to investigate suitable parameters, including silicone moulding, ultrasound, optical three-dimensional profilimetry and high resolution Magnetic Resonance Imaging. All such measurements are readily captured and quantified.

Inhibition of scarring may be demonstrated by a reduction in the height, width, area, perimeter or volume (or any combination thereof), of a treated scar as compared to an untreated scar.

Appearance and/or Colour of Scar Compared to Unscarred Skin.

The appearance or colour of a treated scar may be compared to that of unscarred skin, and/or untreated or control scars and unscarred skin. The appearance of a scar may be compared with unscarred skin with reference to whether the scar is lighter or darker, or redder, than the unscarred skin.

The appearance or colour of the scars and skin may be categorised (e.g. perfectly matched to one another, slightly mismatched, obviously mismatched or grossly mismatched) and these categorisations recorded and/or quantified. Suitable comparisons may be made on the basis of a visual assessment of the respective scars and unscarred skin.

Alternatively or additionally to visual assessment, there are a number of non-invasive colorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope. The results obtained using such devices may also be recorded and quantified.

### Scar Distortion and Mechanical Performance

Scar distortion may be assessed by visual comparison of a scar and unscarred skin, and the degree of distortion categorised (e.g. as causing no distortion, mild distortion, moderate distortion or severe distortion).

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer.

Categorisation information, or values obtained using suitable devices, may be recorded and quantified as required.

### Scar Contour and Scar Texture

Scar contour may be investigated by means of visual assessment, and the contour and texture categorised using suitable parameters. Suitable parameters for categorisation may include whether or not a scar is flush with surrounding skin, slightly proud, slightly indented, hypertrophic or keloid. The texture of a scar may be assessed with reference to the scar's appearance, and this may also be undertaken by a visual assessment and categorised accordingly (for example, whether a scar is matt or shiny, or has a roughened or smooth appearance as compared to unscarred skin).

Scar texture may additionally be assessed, and this assessment categorised with reference to whether the scar has the same texture as unscarred skin (normal texture), is just palpable, firm or hard compared to unscarred skin. The texture of scars may also be assessed with reference to the Hamilton scale (described in Crowe et al, 1998).

In addition to the techniques set out above, there are a number of non-invasive profilimetry devices that use optical or mechanical methods for assessment of scar contour and/or texture. Such assessments may be carried out directly or indirectly. Assessments in this manner may give rise to a representative value by which the assessment may be readily captured.

### Photographic Assessments

Photographic assessment of treated and untreated scars may be performed by any suitable assessor. Examples of suitable assessors include independent lay or expert panels, clinicians, or the patients themselves. Treated or untreated scars may be assessed compared to standardised and calibrated photographs of scars.

Scars may be assessed by a trained clinical or independent lay panel to provide categorical ranking data (e.g. that a given treated scar is “better”, “worse” or “no different” when compared to an untreated scar) and/or quantitative data (such as by using a VAS) as described elsewhere in the specification. The capture of these data may make use of suitable software and/or electronic system(s) as described in the applicant's co-pending patent application filed as PCT/GB2005/004787.

Suitable assessments may consider differences in the appearance of a treated scar that occur over time. This may be achieved by comparison of a time-course of images of selected treated and untreated scars. Assessment of the progression of scarring with time may consider changes in the overall appearance of a scar, and/or changes in specific criteria such as those considered elsewhere in the specification (e.g. scar colour, scar texture, scar width).

The assessments and parameters discussed above are suitable for assessment of the effects of an NR4A agonist, such as 6-mercaptopurine, on scar formation, as compared to control, placebo or standard care treatment in animals or humans. It will be appreciated that these assessments and parameters may be utilised in determining a therapeutically effective NR4A agonist that may be used for scar prevention, reduction or inhibition; and in determining therapeutically effective amounts of NR4A agonists, such as 6-mercaptopurine. Appropriate statistical tests may be used to analyse data sets generated from different treatments in order to investigate the significance of results.

Many of the parameters described above for the assessment of scarring have previously been viewed as primarily suitable for the assessment of scarring that results from the healing of a wound. However, the inventors believe that many of these parameters are also suitable for assessment of scarring associated with fibrotic disorders. Additional or alternative parameters that may be considered when assessing scarring associated with fibrotic disorders will be apparent to the skilled person. The following examples are provided by way of illustration only.

Scarring associated with fibrotic disorders may be assessed with reference to trichrome staining (for example Masson's trichrome or Mallory's trichrome) of biopsy samples taken from a tissue believed to be subject to the fibrotic disorder. These samples may be compared with non-scarred tissues that have been taken from tissues not subject to the fibrotic disorder, and with reference tissues representative of staining in the same tissue (or a range of tissues) subject to different extents of scarring associated with the fibrotic disorder. Comparisons of such tissues may allow assessment of the presence and extent of scarring associated with a fibrotic disorder that is present in the tissue of interest. Protocols for trichrome staining are well known to the skilled person, and kits that may be used to conduct trichrome staining are commercially available.

It will be appreciated that in many cases it may be preferred to avoid invasive procedures such as the collection of biopsies. In recognition of this fact a number of non-invasive procedures have been devised that allow assessment of scarring associated with fibrotic disorders without the need for biopsy samples. Examples of such procedures include Fibrotest (FT) and Actitest (AT).

These commercially available assays use five or six biochemical markers of scarring associated with fibrotic disorders for use as a non-invasive alternative to liver biopsy in patients with chronic hepatitis C or B, alcoholic liver disease and metabolic steatosis (for instance the overweight, patients with diabetes or hyperlipidemia). Through use of such biochemical markers, and analysis using selected algorithms, these procedures are able to determine levels of liver fibrosis and necroinflammatory activity. The use of such tests is increasingly clinically accepted as an alternative to biopsies, and the tests are commercially available from suppliers such as BioPredictive.

It will be appreciated by the skilled person that the methods described above may be used to allow assessment of scarring that is associated with one or more fibrotic disorders in order to determine whether or not prevention, reduction or inhibition of such scarring utilising the medicaments or methods of the invention would be advantageous. Furthermore, scar assessment methods of the type described above may be used to determine therapeutically effective NR4A agonists suitable for inhibition of scarring associated with a fibrotic disorder, as well as determining therapeutically effective amounts of an NR4A agonist, such as 6-mercaptopurine.

Successful inhibition of scarring in tendons or ligaments may be indicated by restoration of function of tissues treated with the medicaments or methods of the invention. Suitable indicators of function may include the ability of the tendon or ligament to bear weight, stretch, flex, etc. Such assessments may, for example, be made using electrophysiological reflex examination, surface electromyography, ultrasonography, ultrasound/MRI scan, and self reported symptom and pain questionnaires

The extent of scarring occurring in blood vessels can be measured directly e.g. using ultrasound, or indirectly by means of blood flow. Inhibition of scarring achieved using the medicaments or methods of the invention may lead to a reduction in narrowing of the blood vessel lumen and allow a more normal blood flow.

### Assessment of Accelerated Wound Healing

Assessment of acceleration of wound healing may be made with reference to any suitable index of the healing age of a wound. Such indices may be assessed macroscopically or microscopically as appropriate. It may be preferred that acceleration of wound healing is assessed with reference to the rate at which treated and untreated or control treated wounds re-epithelialise and/or with reference to the rate at which wounds treated and untreated or control wounds contract (for instance as indicated by a decrease in wound width or area).

Acceleration of wound healing achieved using the medicaments or methods of the invention may preferably lead to a treated wound healing at a rate at least 5% faster than an untreated or control wound, preferably at a rate at least 10% faster, more preferably at least 15%, 20% or 25% faster; yet more preferably at least 50% faster, still more preferably at least 75% faster, and most preferably 100% (or more) faster.

Acceleration of healing using the medicaments or methods of the invention may also give rise to a treated wound having an increased “healing age” when compared with an untreated or control treated wound. Such an increase in healing age may be assessed macroscopically, visually or clinically to determine maturity of the treated wound compared to a suitable untreated or control wound. A wound treated with the medicaments or methods of the invention may preferably have a healing age that is 1, 2, 3, 4, 5 or more days greater than that of an untreated, or control treated wound of the same chronological age.

The rate of re-epithelialisation of treated, untreated or control treated wounds may be assessed macroscopically or microscopically. Macroscopic assessment of re-epithelialisation may be performed, directly using measurements taken from wounds themselves, or indirectly, using measurements taken from images or tracings of wounds. Techniques such as image analysis may be used for the quantification of such measurements.

Microscopic assessment of re-epithelialisation represents a preferred method by which acceleration of wound healing may be assessed. Many suitable protocols by which re-epithelialisation may be microscopically assessed will be well known or apparent to those skilled in the art.

Measurements that may be of use in assessing the rate of promotion of re-epithelialisation, and thus acceleration of wound healing, include the rate at which the surface of the wounded site is covered by an epithelial layer after wounding. Accelerated healing achieved using the medicaments or methods of the invention may preferably lead to a treated wound in which the extent of re-epithelialisation increases at a rate at least 5% faster than an untreated or control treated wound, preferably at a rate at least 10% faster, more preferably at least 15%, 20% or 25% faster; yet more preferably at least 50% faster, still more preferably at least 75% faster, and most preferably 100% (or more) faster. Suitable methods by which the extent of wound re-epithelialisation may be measured in order to assess promotion of contraction of wounds are described elsewhere in the specification. A preferred methodology that may be used for the assessment of the rate of re-epithelialisation (and thus acceleration of wound healing) is described in the Experimental Results section.

Additionally or alternatively, the area of a wound may be assessed macroscopically or microscopically in order to determine the rate of wound healing. Suitable assessments of wound area may, for example, utilise photographs or tracings of the wound margins. These may be considered over time, or with reference to standard comparison data, to assess whether or not wound area is being therapeutically decreased.

Suitable methods by which wound width may be measured in order to assess promotion of wound contraction are described elsewhere in the specification. It may generally be preferred that wound width be assessed microscopically, using histological slides. A preferred protocol for the assessment of wound width in full thickness wounds involves assessing the width of the wound at its mid point (i.e. a point mid way into the depth of the wound). The mid point is preferably in the dermis of the wound, well below the level at which re-epithelialisation occurs. Measurement of wound width at this point may avoid any inaccuracies that may otherwise be arise if wound width is not clearly

A preferred measurement that may be used in assessing acceleration of wound healing is the rate at which the width of a wound decreases. Acceleration of wound healing using the medicaments or methods of the invention may preferably lead to a treated wound in which wound width decreases at a rate at least 5% faster than a control or untreated wound, preferably at a rate at least 10% faster, more preferably at least 15%, 20% or 25% faster; yet more preferably at least 50% faster, still more preferably at least 75% faster, and most preferably 100% (or more) faster.

### Administration Regimes

The methods or medicaments of the invention may be used to provide a therapeutically effective amount of an NR4A agonist, such as 6-mercaptopurine, to a site of existing scarring (whether as a result of a wound or fibrotic disorder), or to a site where scarring is likely to occur (for example a wound, or site of a fibrotic disorder, or a site where a wound or fibrotic disorder is likely to occur). Alternatively, the medicaments or methods of the invention may be used prophylactically, i.e. prior to scar formation. For example, methods or medicaments of the invention may be utilised prior to wounding or prior to the onset of a fibrotic disorder.

In the case of the inhibition of scarring associated with healing of a wound, prophylactic use may involve administration of a therapeutically effective amount of an NR4A agonist, such as 6-mercaptopurine, at sites where no wound presently exists, but where a wound that would otherwise give rise to a scar is to be formed. By way of example, a therapeutically effective amount of an NR4A agonist, such as 6-mercaptopurine, may be administered to sites that are to undergo wounding as a result of elective procedures (such as surgery), or to sites that are believed to be at elevated risk of wounding.

It may be preferred that the medicaments of the invention are administered to the site around the time of wounding, or immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring). The prophylactic use of methods and medicaments in accordance with the invention is a preferred embodiment of the invention, and is particularly preferred in the prevention, reduction or inhibition of scarring in the context of surgical wounds.

In the case of the inhibition of scarring associated with fibrotic disorders, medicaments of the invention may be administered to a site at elevated risk of developing a fibrotic disorder prior to formation of said disorder. Suitable sites may be those that are perceived to be at elevated risk of the development of fibrotic disorders. An elevated risk of development of fibrotic disorders may arise as a result of disease, or as a result of environmental factors (including exposure to fibrotic agents), or as a result of genetic predisposition.

When used for the inhibition of scarring associated with fibrotic disorder, a therapeutically effective amount of an NR4A agonist, such as 6-mercaptopurine, may be administered immediately prior to onset of a fibrotic disorder, or at an earlier time. The skilled person will be able to establish the optimal time for administration of medicaments of the invention used to treat fibrotic disorders using standard techniques well known to those skilled in the art, and familiarisation with the clinical progression of scarring associated with fibrotic disorders.

The methods and medicaments of the invention are also able to inhibit scarring if administered after a wound has already been formed. It is preferred that such administration should occur as early as possible after formation of the wound, but agents of the invention are able to inhibit scarring at any time up until the healing process has been completed (i.e. even in the event that a wound has already partially healed the methods and medicaments of the invention may be used to inhibit scarring in respect of the remaining un-healed portion). It will be appreciated that the “window” in which the methods and medicaments of the invention may be used to inhibit scarring is dependent on the nature of the wound in question (including the degree of damage that has occurred, and the size of the wounded area). Thus, in the case of a large wound, the methods and medicaments of the invention may be administered relatively late in the healing response yet still be able to inhibit scarring, as a consequence of the relatively prolonged time that large wounds require to heal.

The methods and medicaments of the invention may, for instance, preferably be administered within the first 24 hours after a wound is formed, but may still inhibit scarring if administered up to ten, or more, days after wounding.

Similarly, the methods and medicaments of the invention may be administered to a site at which a fibrotic disorder is already developing, in order to prevent further scarring associated with the fibrotic disorder taking place. This use will obviously be advantageous in situations in which the degree of scarring that has occurred prior to administration of an NR4A agonist, such as 6-mercaptopurine, is sufficiently low that the fibrotic tissue is still able to function.

Medicaments of the invention may preferably be administered within 24 hours of the onset of scarring associated with a fibrotic disorder, but may still be effective if administered considerably later in the fibrotic process. For example, medicaments of the invention may be administered within a month of the onset of the fibrotic disorder (or of the diagnosis that scarring associated with the fibrotic disorder is taking place), or within sixth months, or even one or more years, depending on the extent of scarring that has already occurred, the proportion of the tissue effected by the fibrotic disorder, and the rate at which the fibrotic disorder is progressing.

The methods and medicaments of the invention may be administered on one or more occasions (as necessary) in order to inhibit scarring.

For instance, in the case of inhibition of scarring that results from the healing of a wound, therapeutically effective amounts of NR4A agonists, such as 6-mercaptopurine, may be administered to a wound as often as required until the healing process has been completed. By way of example, the medicaments of the invention may be administered daily or twice daily to a wound for at least the first three days following the formation of the wound. In a particularly preferred embodiment a medicament of the invention may be administered prior to wounding and again approximately 24 hours following wounding.

Most preferably the methods or medicaments of the invention may be administered both before and after formation of a wound. The inventors have found that administration of the medicaments of the invention immediately prior to the formation of a wound, followed by daily administration of an NR4A agonist, such as 6-mercaptopurine, for one or more days following wounding, is particularly effective in inhibiting scarring resulting from the healing of a wound, or associated with a fibrotic disorder.

In the case where an NR4A agonist, such as 6-mercaptopurine, is to be used to inhibit scarring associated with a fibrotic disorder, a therapeutically effective amount of an NR4A agonist, may be provided by means of a number of administrations. Suitable regimes may involve administration monthly, weekly, daily or twice daily.

The inventors believe that therapeutically effective amounts of NR4A agonists, such as 6-mercaptopurine, may also be used to reduce existing scars. This is applicable to existing scars that result from the healing of a wound, and/or existing scars associated with fibrotic disorders. Accordingly the use of methods and medicaments of the invention in the reduction of existing scars constitutes a preferred use according to the invention. A therapeutically effective amount of an NR4A agonist, such as 6-mercaptopurine, may be provided by means of any number of suitable administrations. Suitable regimes for these administrations may be readily devised by the skilled person using techniques (including in vitro studies, animal and human studies) well known and established within the pharmaceutical industry.

It will be appreciated that the amount of a medicament of the invention that should be provided to a wound or fibrotic disorder, in order that a therapeutically effective amount of an active agent may be administered, depends on a number of factors. These include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the active agent in the subject being treated.
B) The specific condition to be treated (e.g. acute wounding or chronic fibrotic disorders).
C) The age of the subject.
D) The size of the site to be treated.

The frequency of administration will also be influenced by the above-mentioned factors and particularly the half-life of the chosen agent within the subject being treated.

Generally when medicaments in accordance with the invention are used to treat existing scars (whether resulting from the healing of a wound, or associated with a fibrotic disorder) the medicament should be administered as early as possible in the scarring process or as the fibrotic disorder begins. In the case of wounds or fibrotic disorders that are not immediately apparent, such as those at internal body sites, medicaments may be administered as soon as the wound or disorder, and hence the risk of scarring, is diagnosed. Therapy with methods or medicaments in accordance with the invention should continue until scarring has been inhibited to a clinician's satisfaction.

Frequency of administration will depend upon the biological half-life of the agent used. Typically a cream or ointment containing an agent of the invention should be administered to a target tissue such that the concentration of the agent at a wound or site of fibrosis is maintained at a level suitable to inhibit scarring. This may require administration daily or even several times daily. The inventors have found that administration of an agent of the invention immediately prior to wounding, with a further administration one day after wounding is particularly effective for the inhibition of scarring that would otherwise result from the healing of such a wound.

Daily doses of an agent of the invention may be given as a single administration (e.g. a daily application of a topical formulation or a daily injection). Alternatively, the agent of the invention may require administration twice or more times during a day. In a further alternative, a slow release device may be used to provide optimal doses of an agent of the invention to a patient without the need to administer repeated doses.

### Routes of Administration

Therapeutically effective amounts of NR4A agonists, such as 6-mercaptopurine, may be administered by any suitable route capable of achieving the desired effect of inhibiting scarring. However, it may generally be preferred that an NR4A agonist, such as 6-mercaptopurine, is provided to a tissue, the scarring of which is to be inhibited, by local administration.

Suitable methods by which such local administration may be achieved will depend on the identity of the tissue or organ in question, and may also be influenced by whether the scarring to be inhibited is scarring resulting from the healing of a wound, or scarring associated with a fibrotic disorder. The selection of preferred routes of administration may also depend on whether or not a tissue or organ to be treated is permeable to the chosen medicament. Suitable routes of administration may be selected from the group consisting of: injections; application of sprays, ointments, or creams; inhalation of medicaments; release from biomaterials or other solid medicaments including sutures or wound dressings. Generally, preferred routes of administration may include local injection (for example intradermal injection in the case where it is wished to inhibit scarring of the skin). Suitable formulations for use in these embodiments of the invention are considered elsewhere in the specification.

Medicaments of the invention may be administered in a topical form to inhibit scarring (whether resulting from the healing of a wound or associated with a fibrotic disorder). In the case of inhibiting scarring that would otherwise result from the healing of a wound, such administration may be effected as part of the initial and/or follow up care for the wounded area. Injections may be administered around the margins of a wound, or a site of fibrosis. In the case of their prophylactic use, medicaments of the invention may be applied to a site where a wound or fibrotic disorder will occur.

In the case where it is wished to provide a therapeutically effective amount of an NR4A agonist, such as 6-mercaptopurine, to internal wounds such as those caused by surgical procedures (which may otherwise be prone to formation of adhesions), medicaments comprising an NR4A agonist may be administered by lavage, or by means of a parenteral gel/instillate, or by means of release from local devices (such as sutures, films or carriers able to release NR4A agonists to their surroundings) that may be inserted at the time of surgery.

In the case of scarring of blood vessels, suitable routes of administration may include direct injection into the walls of the blood vessel (for instance before suturing), bathing an anastomotic site in a medium comprising an NR4A agonist, such as 6-mercaptopurine, or administration of an agonist by local applied devices, e.g. sutures or stents. Effective inhibition of scarring in blood vessels may be indicated by the maintenance of a normal level of blood flow following blood vessel injury.

Scarring associated with fibrotic disorders will frequently occur in relatively inaccessible tissues and organs, and it may be preferred that when scarring associated with a fibrotic disorder is to be inhibited an NR4A agonist, such as 6-mercaptopurine, is administered systemically. Suitable routes of administration include, without limitation, oral, transdermal, inhalation, parenteral, sublingual, rectal, vaginal and intranasal. By way of example, solid oral formulations (such as tablets or capsules) providing a therapeutically effective amount of an NR4A agonist, such as 6-mercaptopurine, may be used for the inhibition of scarring associated with renal fibrosis or cirrhosis of the liver. Aerosol formulations for inhalation may be preferred as means for providing NR4A agonists, such as 6-mercaptopurine, in the event that it is wished to inhibit scarring associated with chronic obstructive pulmonary disease or other fibrotic disorders of the lungs and airways.

It will be appreciated that some of the routes of administration described above with reference to systemic administration may also be suitable for localised or topical administration to a tissue in which it is wished to inhibit scarring (for example, inhalation or intranasal administration for inhibition of scarring in the respiratory system, whether as a result of the healing of a wound, or associated with a fibrotic disorder).

Preferred Formulations for Use in Accordance with the Invention

Generally, medicaments of the invention may be formulated and manufactured in any form that allows for the medicament to be administered to a patient such that a therapeutically effective amount of an NR4A agonist, such as 6-mercaptopurine, is provided to a site where scarring is to be prevented, reduced or inhibited.

Medicaments of the invention may preferably be provided in the form of one of more dosage units providing a therapeutically effective amount (or a known fraction or multiple of a therapeutically effective amount) of an NR4A agonist, such as 6-mercaptopurine. Methods of preparing such dosage units will be well known to the skilled person; for example see Remington's Pharmaceutical Sciences 18^{th} Ed. (1990).

Compositions or medicaments containing active agents may take a number of different forms depending, in particular, on the manner in which they are to be used. Thus, for example, they may be in the form of a liquid, ointment, cream, gel, hydrogel, powder or aerosol. All of such compositions are suitable for topical application to a site of scarring (for example, either a wound or a fibrotic disorder), and topical application represents a preferred means of administering NR4A agonists to a subject (person or animal) in need of treatment. Liquid, gel or hydrogel medicaments containing active agents may be formulated so that they are suitable for administration by local injection, another preferred means by which NR4A agonists such as 6-mercaptopurine may be administered.

Suitable agonists may be provided on a sterile dressing or patch, which may be used to cover a wound or fibrotic site where scarring is to be inhibited.

NR4A agonists, such as 6-mercaptopurine, may be released from a device or implant, or may be used to coat such a device e.g. a stent, or a controlled release device, or a wound dressing, or sutures for use in wound closure.

It will be appreciated that the vehicle of a composition comprising an NR4A agonist, such as 6-mercaptopurine, should be one that is well tolerated by the patient and allows release of the agonist to the wound or fibrotic site. Such a vehicle is preferably biodegradeable, bioresolveable, bioresorbable and/or non-inflammatory.

If the composition is to be applied to an existing wound or fibrotic site, then the pharmaceutically acceptable vehicle will be one which is relatively “mild” i.e. a vehicle which is biocompatible, biodegradable, bioresolvable and non-inflammatory.

NR4A agonists, such as 6-mercaptopurine, may be incorporated within a slow or delayed release device. Such devices may, for example, be placed on or inserted under the skin and the agonist may be released over days, weeks or even months.

Delayed release devices may be particularly useful for patients, such as those suffering from extensive scarring or from long-lasting scarring associated with a fibrotic disorder, who require long-term administration of therapeutically effective amounts of an NR4A agonist, such as 6-mercaptopurine. Such devices may be particularly advantageous when used for the administration of an agonist that would otherwise normally require frequent administration, e.g. at least daily administration, by other routes.

A dose of a composition comprising an NR4A agonist, such as 6-mercaptopurine, may preferably be sufficient to provide a therapeutically effective amount of a suitable agonist in a single administration. However, it will be appreciated that each dose need not in itself provide a therapeutically effective amount of an agonist, but that a therapeutically effective amount may instead be built up through repeated administration of suitable doses.

Various suitable forms of compositions comprising NR4A agonists, such as 6-mercaptopurine, may be used in accordance with the present invention. In one embodiment a pharmaceutical vehicle for administration of a suitable agonist may be a liquid and a suitable pharmaceutical composition would be in the form of a solution. In another embodiment, the pharmaceutically acceptable vehicle is a solid and a suitable composition is in the form of a powder. In a further embodiment the NR4A agonist may be formulated as a part of a pharmaceutically acceptable trans-epidermal delivery system, e.g. a patch/dressing

A solid vehicle can include one or more substances that may also act as flavouring agents, lubricants, solubilizers, suspending agents, fillers, glidants, compression aids, binders or tablet-disintegrating agents; it can also comprise an encapsulating material. In powders, the vehicle is a finely divided solid that is in admixture with the finely divided agonist that will be used to inhibit scarring. In tablets, the selected agonist is mixed with a vehicle having the necessary compression properties in suitable proportions and compacted in the shape and size desired. The powders and tablets preferably contain up to 99% of an NR4A agonist such as 6-mercaptopurine. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The NR4A agonist can be dissolved or suspended in a pharmaceutically acceptable liquid vehicle such as water, an organic solvent, a mixture of both or pharmaceutically acceptable oils or fats. Phosphate buffered saline (PBS) represents an example of a preferred liquid vehicle. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can be an oily ester such as ethyl oleate and isopropyl myristate. Sterile liquid vehicles are useful in compositions for parenteral administration. The liquid vehicle for pressurized compositions can be halogenated hydrocarbon or other pharmaceutically acceptable propellant.

Liquid pharmaceutical compositions which are sterile solutions or suspensions comprising an NR4A agonist, such as 6-mercaptopurine, can be utilized by, for example, intramuscular, intrathecal, epidural, intraperitoneal, intradermal, intraadventitial (blood vessels) or subcutaneous injection. Sterile solutions can also be administered intravenously. The agonist may be prepared as part of a sterile solid composition that may be dissolved or suspended at the time of administration using sterile water, saline, or other appropriate sterile injectable medium (such as PBS). Vehicles are intended to include necessary and inert binders, suspending agents, lubricants and preservatives. The inventors have found that a sterile solution of an NR4A agonist, such as 6-mercaptopurine, in phosphate buffered saline (PBS) containing 5% ethanol represents a preferred formulation suitable for administration by injection, such as intradermal injection.

In the situation in which it is desired to administer an NR4A agonist by means of oral ingestion, it will be appreciated that the chosen agonist will preferably be one having an elevated degree of resistance to degradation. For example, the chosen agonist may be protected (using the techniques well known to those skilled in the art) so that its rate of degradation in the digestive tract is reduced.

Medicaments comprising an NR4A agonist, such as 6-mercaptopurine, that are for use in the inhibition of scarring in the lungs or other respiratory tissues may be formulated for inhalation.

Medicaments in accordance with the invention for use in the inhibition of scarring in the body cavities e.g. abdomen or pelvis, may be formulated as an irrigation fluid, lavage, gel or instillate.

An NR4A agonist, such as 6-mercaptopurine, for use in the medicaments or methods of the invention, may be incorporated in a biomaterial, from which it may be released to inhibit scarring. Biomaterials incorporating NR4A agonists are suitable for use in many contexts, and at many body sites, where it is desired to inhibit scarring, but may be of particular utility in providing a suitable agonist of NR4A, such as 6-mercaptopurine, to sites where it is wished to inhibit restenosis or adhesions. The inventors believe that biomaterials incorporating NR4A agonists, such as 6-mercaptopurine, may be used in the manufacture of sutures, and such sutures represent a preferred embodiment of a medicament of the invention.

Known procedures, such as those conventionally employed by the pharmaceutical industry (e.g. in vivo experimentation, clinical trials etc), may be used to establish specific formulations of compositions comprising NR4A agonists, such as 6-mercaptopurine, and precise therapeutic regimes for administration of such compositions (such as the required daily doses of the agonist, and the preferred frequency of administration).

Medicaments or methods of the invention may be used to inhibit scarring as a monotherapy (e.g. through use of medicaments or methods of the invention alone). Alternatively the methods or medicaments of the invention may be used in combination with other compounds or treatments for the inhibition of scarring. Suitable compounds that may be used as parts of such combination therapies will be well known to those skilled in the art.

It will be appreciated that many of the advantages that may be gained as a result of inhibiting scarring of humans are also applicable to other animals, particularly veterinary or domestic animals (e.g. horses, cattle, dogs, cats etc). Accordingly it will be recognised that the medicaments and methods of the invention may also be used inhibit scarring of non-human animals.

The invention will now be further described with reference to the accompanying Sequence Information, Experimental Results, and Figures, in which:

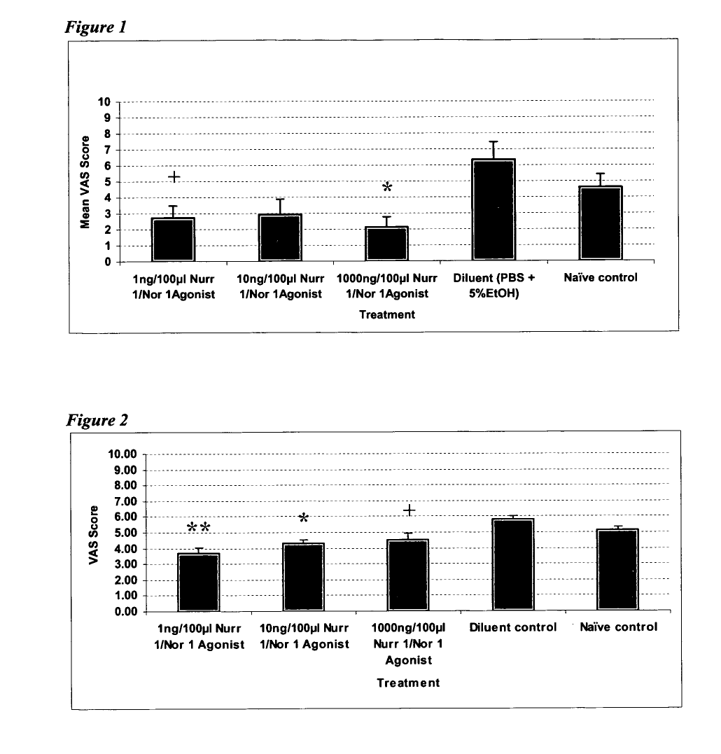

FIG. 1 is a bar graph and compares macroscopic Visual Analogue Scale (VAS) scores, indicative of the level of scarring in treated scars produced on healing of wounds treated with the NR4A agonist 6-mercaptopurine, with macroscopic VAS scores from scars produced on the healing of diluent control wounds and naïve control wounds (* indicates p<0.05 compared to diluent and naïve controls; +indicates p<0.05 compared to diluent controls only), all assessed 70 days after wounding.

FIG. 2 is a bar graph and compares microscopic Visual Analogue Scale (VAS) scores, indicative of the level of scarring in treated scars produced on healing of wounds treated with the NR4A agonist 6-mercaptopurine, with microscopic VAS scores from scars produced on the healing of diluent control wounds and naïve control wounds (** indicates p<0.01 compared to diluent and naïve controls; * indicates p<0.05 compared to diluent and naïve controls; +indicates p<0.05 compared to diluent controls only), all assessed 70 days after wounding.

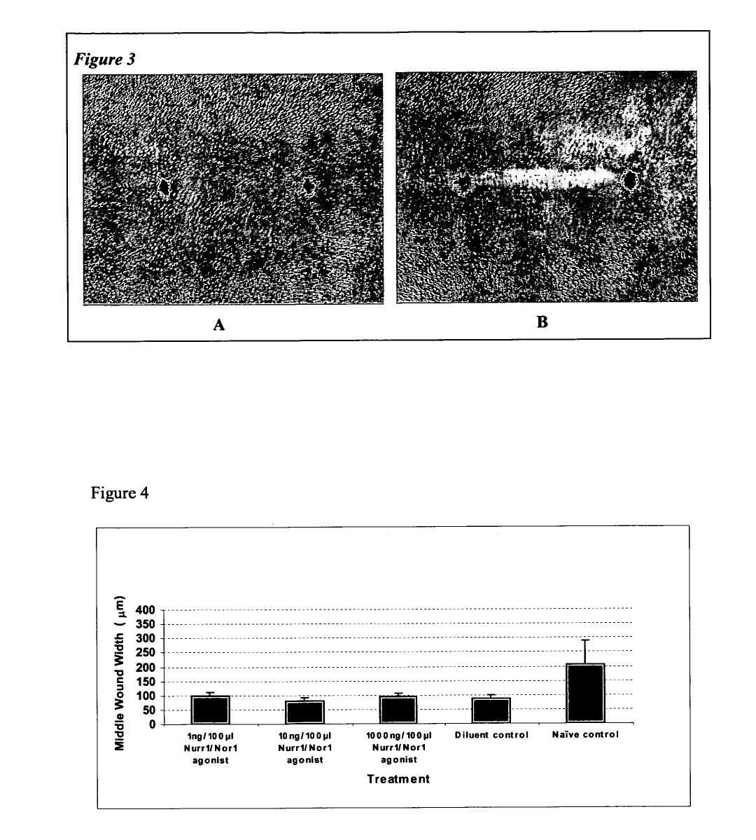

FIG. 3 compares representative images showing the macroscopic appearance of a treated scar (produced on healing of a wound treated with a total of 20 ng of the NR4A agonist 6-mercaptopurine by means of two administrations of a 10 ng/100 μl solution of 6-mercaptopurine), on the left, with a scar produced on the healing of a diluent control wound on the right.

FIG. 4 is a bar graph and compares the width of wounds (measured in micrometers) treated with the NR4A agonist 6-mercaptopurine, with the widths of diluent and naïve control wounds. Widths were assessed by measurement of the middle of the wound three days after surgical incisions were formed.

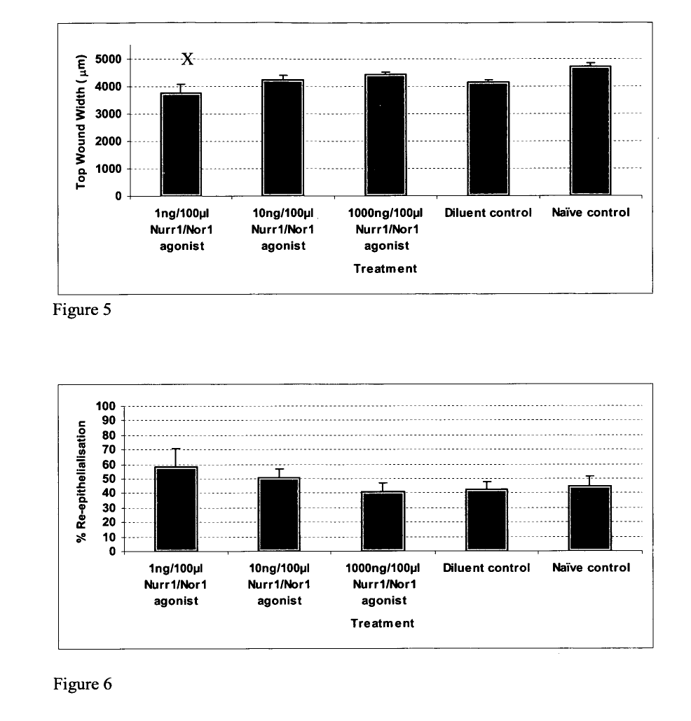

FIG. 5 is a bar graph and compares the widths of excisional wounds treated with the NR4A agonist 6-mercaptopurine with widths of diluent control and naïve control wounds. Width of these excisional wounds was measured across the top of the wound three days after the excision had been formed (X indicates p<0.01 versus naïve control only).

FIG. 6 is a bar graph and compares percentage re-epithelialisation occurring in excisional wounds treated with the NR4A agonist 6-mercaptopurine with the degree of re-epithelialisation occurring in diluent control and naïve control wounds. Percentage re-epithelialisation in these excisional wounds was assessed three days after excisions had been formed.

### EXPERIMENTAL RESULTS

### Materials

The NR4A agonist 6-mercaptopurine monohydrate (Sigma Aldrich, catalogue number 852678) was diluted in phosphate buffered saline (PBS) containing 5% ethanol to produce medicaments of the invention having the following concentrations:

i) 1 ng/100 μL (a concentration of 59 nM, in which each 100 μl of the medicament provides 5.9 pmol of the NR4A agonist 6-mercaptopurine);
ii) 10 ng/100 μL (a concentration of 588 nM, in which each 100 μl of the medicament provides 58.8 pmol of the NR4A agonist 6-mercaptopurine); and
iii) 1000 ng/100 μL (a concentration of 59 μM, in which each 100 μl of the medicament provides 5.9 nmol of the NR4A agonist 6-mercaptopurine).

### Scarring Model

At day 0, male Sprague Dawley rats (200-250 g) were anaesthetised, shaved and wound sites were marked according to the Renovo rat incisional wounding template (2 wound model, 2×1 cm wounds, 5 cm from the base of the skull and 1 cm from the midline in each rat). One hundred microlitres of 6-mercaptopurine at 1 ng, 10 ng or 1000 ng in phosphate buffered saline (PBS, pH 7.2; GIBCO BRL, Cat. #20012-019) containing 5% ethanol was injected intradermally at the wound sites. The intradermal injections caused the formation of a raised bleb, which was then immediately incised to form 1 cm long experimental wounds. Separate groups of rats used as naïve controls and diluent controls. Naïve control animals were wounded without treatment, whilst diluent control animals were injected with 100 μl of the PBS/ethanol mixture prior to wounding. All treated or diluent control wounds were re-injected again 1 day post-wounding with the appropriate treatment via injection of 50 μl to each of the two margins of the 1 cm wound. Wounds from the treated, naïve control and diluent control animals were all harvested at day 70 post-wounding.

### Incisional Wound Healing Model

Treated and control incisional wounds were created as described above, to allow assessment of the effects of NR4A agonists, such as 6-mercaptopurines, on wound healing. Wounds from the treated, naïve control and diluent control animals were all harvested at three days post-wounding.

Excisional Wound Healing Model

At day 0, male Sprague Dawley rats (200-250 g) were anaesthetised, shaved and wound sites were marked according to the Renovo rat excisional wounding template (2 wound model, 2×5 mm punch biopsy excisional wounds, 8.5 cm from the base of the skull and 1 cm from the midline in each rat). One hundred microlitres of 6-mercaptopurine at 1 ng, 10 ng or 1000 ng in phosphate buffered saline (PBS, pH 7.2; GIBCO BRL, Cat. #20012-019) containing 5% ethanol was injected intradermally at the wound sites. The intradermal injections caused the formation of a raised bleb, which was then immediately biopsied to form 5 mm experimental excisional wounds. Separate groups of rats used as naïve controls and diluent controls. Naïve control animals were wounded without treatment, whilst diluent control animals were injected with 100 μl of the PBS/ethanol mixture prior to wounding. All treated or diluent wounds were re-injected again 1 day post-wounding with the appropriate treatment via injection of 25 μl to each quadrant of the margin of the 5 mm excisional wound. Wounds from the treated, naïve control and diluent control animals were all harvested at day 3 post-wounding.

### Assessment of Scarring

The wounds were photographed after wounding, prior to re-injection on day 1 and on day of harvest. The scars were assessed using standard macroscopic scar assessment using a visual analogue scale (VAS) consisting of a 0-10 cm line representing a scale, from left to right, of 0 (corresponding to normal skin) to 10 (indicative of a bad scar). For microscopic assessment the scars were excised from the experimental rats (incorporating a small amount of surrounding normal tissue) and fixed in 10% (v/v) buffered formal saline. The fixed tissue was then processed for wax histology, stained using Masson's trichrome, and scarring assessed using a microscopic visual analogue scale (VAS).

### Assessment of Wound Healing

### Wound Width of Incisional and Excisional Wounds

The wounds were photographed after wounding, prior to re-injection on day one and on the day of harvest. Day three incisional or excisional wounds were excised from the experimental rats (incorporating a small amount of surrounding normal tissue). For microscopic assessment the scars were excised from the experimental rats (incorporating a small amount of surrounding normal tissue) and fixed in 10% (v/v) buffered formal saline. The fixed tissue was then processed for wax histology, stained using Masson's trichrome, and wound width assessed using image analysis software. Width of incisional wounds was measured at a point mid-way through the depth of the wound, whereas width of excisional wounds was measured at the top of the wounds.

### Percentage Re-Epithelialisation of Excisional Wounds

The wounds were photographed after wounding, prior to re-injection on day one and on the day of harvest. Day three excisional wounds were excised from the experimental rats (incorporating a small amount of surrounding normal tissue). For microscopic assessment the scars were excised from the experimental rats (incorporating a small amount of surrounding normal tissue) and fixed in 10% (v/v) buffered formal saline. The fixed tissue was then processed for wax histology, stained using Masson's trichrome, and re-epithelialisation assessed by measuring the epithelial covering present as a percentage of the total wound widths. Both the extent of epithelial coverage and total wound width were assessed using image analysis software.

### Results

Intradermal injections of 6-mercaptopurine at 1 ng/100 μl, 10 ng/100 μl and 1000 ng/100 μl improved the macroscopic appearance of scars resulting from full thickness cutaneous incisional wounds, in comparison to scars resulting from naïve control or diluent control wounds. The 1 ng/100 μl and 1000 ng/100 μl doses of 6-mercaptopurine resulted in a statistically significant inhibition of scarring as assessed with reference to the macroscopic appearance of treated wounds (respectively p<0.05 as compared to diluent controls; and p<0.05 as compared to both diluent and naïve controls).

Intradermal injections of 6-mercaptopurine at concentrations of 1 ng/100 μl, 10 ng/100 μl and 1000 ng/100 μl also significantly improved the microscopic appearance of scars resulting from full thickness cutaneous incisional wounds, in comparison to scars resulting from naïve control or diluent control wounds. 1 ng/100 μl significantly inhibited scarring as compared to both diluent and control wounds (p<0.01); 10 ng/100 μl significantly inhibited scarring as compared to diluent and naïve controls (p<0.05); and 1000 ng/100 μl significantly inhibited scarring as compared to diluent controls (p<0.05).

Surprisingly, the results obtained indicate that injections of 6-mercaptopurine did not inhibit wound healing. The widths of day 3 incisional or excisional wounds were not increased as compared to controls (as would be expected if 6-mercaptopurine was inhibiting wound healing) and, in fact, were consistently lower than those in naïve controls (significantly so, in the case of excisional wounds receiving with 1 ng/100 μl treatment). The major factor influencing wound width is thought to be wound contraction. The contractile force responsible is brought about by myofibroblasts in or around the wounded area. The fact that treated wounds were not subject to any impairment of wound contraction (and actually demonstrated improved closure) indicates that the effective doses are not impairing fibroblast function.

As well as exhibiting increased wound contraction, the treated wounds showed a trend towards increased re-epithelialisation. Three days after wounding the incisional wounds were fully re-epithelialised (comparable with control wounds) indicating that treatment with 6-mercaptopurine did not impair wound closure. In excisional wounds, where the process of re-epithelialisation takes longer, due to the larger area of the wounds involved, wounds treated with 6-mercaptopurine actually showed a trend towards increased re-epithelialisation. This indicates that the doses of 6-mercaptopurine that are used are not impairing keratinocyte function.

These results clearly illustrate the ability of NR4A agonists, such as 6-mercaptopurine, to prevent, reduce or inhibit scarring in non-ocular tissues in vivo, when administered in a therapeutically effective amount. Furthermore, these agonists demonstrate the ability to accelerate wound healing, either by increasing wound contraction, or increasing re-epithelialisation.

### Sequence Information

Nuclear Receptor Subfamily 4, Group A, Member 1 [Homo sapiens].
Sequence ID No. 1—Amino Acid sequence (NP_{—}002126):

| | |
|---|---|
| 1 | mpciqaqygt papspgprdh lasdpltpef ikptmdlasp eaapaaptal psfstfmdgy |
| 61 | tgefdtflyq lpgtvqpcss asssasstss ssatspasas fkfedfqvyg cypgplsgpv |
| 121 | dealsssgsd yygspcsaps pstpsfqppq lspwdgsfgh fspsqtyegl rawteqlpka |
| 181 | sgppqppaff sfspptgpsp slaqsplklf psqathqlge gesysmptaf pglaptsphl |
| 241 | egsgildtpv tstkarsgap ggsegrcavc gdnascqhyg vrtcegckgf fkrtvqknak |
| 301 | yiclankdcp vdkrrrnrcq fcrfqkclav gmvkevvrtd slkgrrgrlp skpkqppdas |
| 361 | panlltslvr ahldsgpsta kldyskfqel vlphfgkeda gdvqqfydll sgslevirkw |
| 421 | aekipgfael spadqdllle saflelfilr layrskpgeg klifcsglvl hrlqcargfg |
| 481 | dwidsilafs rslhsllvdv pafaclsalv litdrhglqe prrveelqnr iasclkehva |
| 541 | avagepqpas clsrllgklp elrtlctqgl qrifylkled lvppppiidk ifmdtlpf |

### Sequence ID No. 4—Nucleotide Sequence (NM_{—}002135):

Based on transcript variant 1 mRNA; variant 1 (this variant) represents the longer transcript. Both variants 1 and 2 encode the same protein.

| | |
|---|---|
| 1 | ggagcgctta agaggagggt cgggctcggc cggggagtcc cagtggcgga ggctacgaaa |
| 61 | cttgggggag tgcacagaag aacttcggga gcgcacgcgg gaccagggac caggctgaga |
| 121 | ctcggggcgc cagtccgggc aggggcagcg ggagccggcc gggtagggtg cagcctgagg |
| 181 | cttgttcagc agaacaggtg caagccacat tgttgccaag acctgcctga agccggattc |
| 241 | tccccactgc ctccttcaac cccgcctctt cctcctcctg tgggactgct cccccctcct |
| 301 | gtgaggctag atagatgccc tgtatccaag cccaatatgg gacaccagca ccgagtccgg |
| 361 | gaccccgtga ccacctggca agcgaccccc tgacccctga gttcatcaag cccaccatgg |
| 421 | acctggccag ccccgaggca gcccccgctg cccccactgc cctgcccagc ttcagcacct |
| 481 | tcatggacgg ctacacagga gagtttgaca ccttcctcta ccagctgcca ggaacagtcc |
| 541 | agccatgctc ctcagcctcc tcctcggcct cctccacatc ctcgtcctca gccacctccc |
| 601 | ctgcctctgc ctccttcaag ttcgaggact tccaggtgta cggctgctac cccggccccc |
| 661 | tgagcggccc agtggatgag gccctgtcct ccagtggctc tgactactat ggcagcccct |
| 721 | gctcggcccc gtcgccctcc acgcccagct tccagccgcc ccagctctct ccctgggatg |
| 781 | gctccttcgg ccacttctcg cccagccaga cttacgaagg cctgcgggca tggacagagc |
| 841 | agctgcccaa agcctctggg cccccacagc ctccagcctt cttttccttc agtcctccca |
| 901 | ccggccccag ccccagcctg gcccagagcc ccctgaagtt gttcccctca caggccaccc |
| 961 | accagctggg ggagggagag agctattcca tgcctacggc cttcccaggt ttggcaccca |
| 1021 | cttctccaca ccttgagggc tcggggatac tggatacacc cgtgacctca accaaggccc |
| 1081 | ggagcggggc cccaggtgga agtgaaggcc gctgtgctgt gtgtggggac aacgcttcat |
| 1141 | gccagcatta tggtgtccgc acatgtgagg gctgcaaggg cttcttcaag cgcacagtgc |
| 1201 | agaaaaacgc caagtacatc tgcctggcta acaaggactg ccctgtggac aagaggcggc |
| 1261 | gaaaccgctg ccagttctgc cgcttccaga agtgcctggc ggtgggcatg gtgaaggaag |
| 1321 | ttgtccgaac agacagcctg aaggggcggc ggggccggct accttcaaaa cccaagcagc |
| 1381 | ccccagatgc ctcccctgcc aatctcctca cttccctggt ccgtgcacac ctggactcag |
| 1441 | ggcccagcac tgccaaactg gactactcca agttccagga gctggtgctg ccccactttg |
| 1501 | ggaaggaaga tgctggggat gtacagcagt tctacgacct gctctccggt tctctggagg |
| 1561 | tcatccgcaa gtgggcggag aagatccctg gctttgctga gctgtcaccg gctgaccagg |
| 1621 | acctgttgct ggagtcggcc ttcctggagc tcttcatcct ccgcctggcg tacaggtcta |
| 1681 | agccaggcga gggcaagctc atcttctgct caggcctggt gctacaccgg ctgcagtgtg |
| 1741 | cccgtggctt cggggactgg attgacagta tcctggcctt ctcaaggtcc ctgcacagct |
| 1801 | tgcttgtcga tgtccctgcc ttcgcctgcc tctctgccct tgtcctcatc accgaccggc |
| 1861 | atgggctgca ggagccgcgg cgggtggagg agctgcagaa ccgcatcgcc agctgcctga |
| 1921 | aggagcacgt ggcagctgtg gcgggcgagc cccagccagc cagctgcctg tcacgtctgt |
| 1981 | tgggcaaact gcccgagctg cggaccctgt gcacccaggg cctgcagcgc atcttctacc |
| 2041 | tcaagctgga ggacttggtg ccccctccac ccatcattga caagatcttc atggacacgc |
| 2101 | tgcccttctg acccctgcct gggaacacgt gtgcacatgc gcactctcat atgccacccc |
| 2161 | atgtgccttt agtccacgga cccccagagc acccccaagc ctgggcttga gctgcagaat |
| 2221 | gactccacct tctcacctgc tccaggaggt ttgcagggag ctcaagccct tggggagggg |
| 2281 | gatgccttca tgggggtgac cccacgattt gtcttatccc ccccagcctg gccccggcct |
| 2341 | ttatgttttt tgtaagataa accgttttta acacatagcg ccgtgctgta aataagccca |
| 2401 | gtgctgctgt aaatacagga agaaagagct tgaggtggga gcggggctgg gaggaaggga |
| 2461 | tgggccccgc cttcctgggc agcctttcca gcctcctgct ggctctctct tcctaccctc |
| 2521 | cttccacatg tacataaact gtcactctag gaagaagaca aatgacagat tctgacattt |
| 2581 | atatttgtgt attttcctgg atttatagta tgtgactttt ctgattaata tatttaatat |
| 2641 | attgaataaa aaatagacat gtagttggaa aaaaaaaaaa aaaaaaaaaa aaaaaaaaa |

Nuclear Receptor Subfamily 4, Group A, Member 2 [Homo sapiens].

Isoforms of this protein exist, the longest of which is isoform A (sequence ID No. 2). Variant transcripts exist, and nucleotide sequence (sequence ID No. 5) represents the longest of these (variant 1), which encodes isoform A.

Sequence ID No. 2—Amino Acid sequence (NP_{—}006177):

| | |
|---|---|
| 1 | mpcvqaqygs spqgaspasq sysyhssgey ssdfltpefv kfsmdltnte itattslpsf |
| 61 | stfmdnystg ydvkppclyq mplsgqqssi kvediqmhny qqhshlppqs eemmphsgsv |
| 121 | yykpsspptp ttpgfqvqhs pmwddpgslh nfhqnyvatt hmieqrktpv srlslfsfkq |
| 181 | sppgtpvssc qmrfdgplhv pmnpepagsh hvvdgqtfav pnpirkpasm gfpglqigha |
| 241 | sqlldtqvps ppsrgspsne glcavcgdna acqhygvrtc egckgffkrt vqknakyvcl |
| 301 | ankncpvdkr rrnrcqycrf qkclavgmvk evvrtdslkg rrgrlpskpk spqepsppsp |
| 361 | pvslisalvr ahvdsnpamt sldysrfqan pdyqmsgddt qhiqqfydll tgsmeiirgw |
| 421 | aekipgfadl pkadqdllfe saflelfvlr layrsnpveg klifcngvvl hrlqcvrgfg |
| 481 | ewidsivefs snlqnmnidi safsciaala mvterhglke pkrveelqnk ivnclkdhvt |
| 541 | fnngglnrpn ylskllgklp elrtlctqgl qrifylkled lvpppaiidk lfldtlpf |

### Sequence ID No. 5—Nucleotide Sequence (NM_{—}006186);

| | |
|---|---|
| 1 | ggccagtccg cccggcggct cgcgcacggc tccgcggtcc cttttgcctg tccagccggc |
| 61 | cgcctgtccc tgctccctcc ctccgtgagg tgtccgggtt cccttcgccc agctctccca |
| 121 | cccctacccg accccggcgc ccgggctccc agagggaact gcacttcggc agagttgaat |
| 181 | gaatgaagag agacgcggag aactcctaag gaggagattg gacaggctgg actccccatt |
| 241 | gcttttctaa aaatcttgga aactttgtcc ttcattgaat tacgacactg tccaccttta |
| 301 | atttcctcga aaacgcctgt aactcggctg aagccatgcc ttgtgttcag gcgcagtatg |
| 361 | ggtcctcgcc tcaaggagcc agccccgctt ctcagagcta cagttaccac tcttcgggag |
| 421 | aatacagctc cgatttctta actccagagt ttgtcaagtt tagcatggac ctcaccaaca |
| 481 | ctgaaatcac tgccaccact tctctcccca gcttcagtac ctttatggac aactacagca |
| 541 | caggctacga cgtcaagcca ccttgcttgt accaaatgcc cctgtccgga cagcagtcct |
| 601 | ccattaaggt agaagacatt cagatgcaca actaccagca acacagccac ctgccccccc |
| 661 | agtctgagga gatgatgccg cactccgggt cggtttacta caagccctcc tcgcccccga |
| 721 | cgcccaccac cccgggcttc caggtgcagc acagccccat gtgggacgac ccgggatctc |
| 781 | tccacaactt ccaccagaac tacgtggcca ctacgcacat gatcgagcag aggaaaacgc |
| 841 | cagtctcccg cctctccctc ttctccttta agcaatcgcc ccctggcacc ccggtgtcta |
| 901 | gttgccagat gcgcttcgac gggcccctgc acgtccccat gaacccggag cccgccggca |
| 961 | gccaccacgt ggtggacggg cagaccttcg ctgtgcccaa ccccattcgc aagcccgcgt |
| 1021 | ccatgggctt cccgggcctg cagatcggcc acgcgtctca gctgctcgac acgcaggtgc |
| 1081 | cctcaccgcc gtcgcggggc tccccctcca acgaggggct gtgcgctgtg tgtggggaca |
| 1141 | acgcggcctg ccaacactac ggcgtgcgca cctgtgaggg ctgcaaaggc ttctttaagc |
| 1201 | gcacagtgca aaaaaatgca aaatacgtgt gtttagcaaa taaaaactgc ccagtggaca |
| 1261 | agcgtcgccg gaatcgctgt cagtactgcc gatttcagaa gtgcctggct gttgggatgg |
| 1321 | tcaaagaagt ggttcgcaca gacagtttaa aaggccggag aggtcgtttg ccctcgaaac |
| 1381 | cgaagagccc acaggagoco tctccccctt cgcccccggt gagtctgatc agtgccctcg |
| 1441 | tcagggccca tgtcgactcc aacccggcta tgaccagcct ggactattcc aggttccagg |
| 1501 | cgaaccctga ctatcaaatg agtggagatg acacccagca tatccagcaa ttctatgatc |
| 1561 | tcctgactgg ctccatggag atcatccggg gctgggcaga gaagatccct ggcttcgcag |
| 1621 | acctgcccaa agccgaccaa gacctgcttt ttgaatcagc tttcttagaa ctgtttgtcc |
| 1681 | ttcgattagc atacaggtcc aacccagtgg agggtaaact catcttttgc aatggggtgg |
| 1741 | tcttgcacag gttgcaatgc gttcgtggct ttggggaatg gattgattcc attgttgaat |
| 1801 | tctcctccaa cttgcagaat atgaacatcg acatttctgc cttctcctgc attgctgccc |
| 1861 | tggctatggt cacagagaga cacgggctca aggaacccaa gagagtggaa gaactgcaaa |
| 1921 | acaagattgt aaattgtctc aaagaccacg tgactttcaa caatgggggg ttgaaccgcc |
| 1981 | ccaattattt gtccaaactg ttggggaagc tcccagaact tcgtaccctt tgcacacagg |
| 2041 | ggctacagcg cattttctac ctgaaattgg aagacttggt gccaccgcca gcaataattg |
| 2101 | acaaactttt cctggacact ttacctttct aagacctcct cccaagcact tcaaaggaac |
| 2161 | tggaatgata atggaaactg tcaagagggg gcaagtcaca tgggcagaga tagccgtgtg |
| 2221 | agcagtctca gctcaagctg ccccccattt ctgtaaccct cctagccccc ttgatcccta |
| 2281 | aagaaaacaa acaaacaaac aaaaactgtt gctatttcct aacctgcagg cagaacctga |
| 2341 | aagggcattt tggctccggg gcatcctgga tttagaacat ggactacaca caatacagtg |
| 2401 | gtataaactt tttattctca gtttaaaaat cagtttgttg ttcagaagaa agattgctat |
| 2461 | aatgtataat gggaaatgtt tggccatgct tggttgttgc agttcagaca aatgtaacac |
| 2521 | acacacacat acacacacac acacacacac agagacacat cttaagggga cccacaagta |
| 2581 | ttgcccttta acaagacttc aaagttttct gctgtaaaga aagctgtaat atatagtaaa |
| 2641 | actaaatgtt gcgtgggtgg catgagttga agaaggcaaa ggcttgtaaa tttacccaat |
| 2701 | gcagtttggc tttttaaatt attttgtgcc tatttatgaa taaatattac aaattctaaa |
| 2761 | agataagtgt gtttgcaaaa aaaaagaaaa taaatacata aaaaagggac aagcatgttg |
| 2821 | attctaggtt gaaaatgtta taggcacttg ctacttcagt aatgtctata ttatataaat |
| 2881 | agtatttcag acactatgta gtctgttaga ttttataaag attggtagtt atctgagctt |
| 2941 | aaacattttc tcaattgtaa aataggtggg cacaagtatt acacatcaga aaatcctgac |
| 3001 | aaaagggaca catagtgttt gtaacaccgt ccaacattcc ttgtttgtaa gtgttgtatg |
| 3061 | taccgttgat gttgataaaa agaaagttta tatcttgatt attttgttgt ctaaagctaa |
| 3121 | acaaaacttg catgcagcag cttttgactg tttccagagt gcttataata tacataactc |
| 3181 | cctggaaata actgagcact ttgaattttt tttatgtcta aaattgtcag ttaatttatt |
| 3241 | attttgtttg agtaagaatt ttaatattgc catattctgt agtatttttc tttgtatatt |
| 3301 | tctagtatgg cacatgatat gagtcactgc ctttttttct atggtgtatg acagttagag |
| 3361 | atgctgattt tttttctgat aaattctttc tttgagaaag acaattttaa tgtttacaac |
| 3421 | aataaaccat gtaaatgaaa aaaaaaa |

Nuclear Receptor Subfamily 4, Group A, Member 3 [Homo sapiens].

Isoforms of this protein exist, the longest of which (isoform A) is shown in Sequence ID No. 3). Variant transcripts encoding the protein exist. Both transcript variants 1 and 2 encode isoform A. Sequence ID No. 6 is transcript variant 1.

Sequence ID No. 3—Amino Acid sequence (NP_{—}008912):

| | |
|---|---|
| 1 | mpcvqaqysp sppgssyaaq tysseyttei mnpdytkltm dlgsteitat attslpsist |
| 61 | fvegyssnye lkpscvyqmq rplikveegr apsyhhhhhh hhhhhhhhqq qhqqpsippa |
| 121 | sspedevlps tsmyfkqspp stpttpafpp qagalwdeal psapgciapg plldppmkav |
| 181 | ptvagarfpl fhfkpspphp papspagghh lgydptaaaa lslplgaaaa agsqaaales |
| 241 | hpyglplakr aaplafpplg ltpsptassl lgespslpsp psrssssgeg tcavcgdnaa |
| 301 | cqhygvrtce gckgffkrtv qknakyvcla nkncpvdkrr rnrcqycrfq kclsvgmvke |
| 361 | vvrtdslkgr rgrlpskpks plqqepsqps ppsppicmmn alvraltdst prdldysryc |
| 421 | ptdqaaagtd aehvqqfynl ltasidvsrs waekipgftd lpkedqtlli esaflelfvl |
| 481 | rlsirsntae dkfvfcnglv lhrlqclrgf gewldsikdf slnlqslnld iqalaclsal |
| 541 | smiterhglk epkrveelcn kitsslkdhq skgqalepte skvlgalvel rkictlglqr |
| 601 | ifylkledlv sppsiidklf ldtlpf |

### Sequence ID No. 6—Nucleotide Sequence (NM_{—}006981):

| | |
|---|---|
| 1 | ataaatgacg tgccgagaga gcgagcgaac gcgcagccgg gagagcggag tctcctgcct |
| 61 | cccgcccccc acccctccag ctcctgctcc tcctccgctc cccatacaca gacgcgctca |
| 121 | cacccgctcc ctcactcgca cacacagaca caagcgcgca cacaggctcc goacacacac |
| 181 | ttcgctctcc cgcgcgctca cacccctctt gccctgagcc cttgccggtg cagcgcggcg |
| 241 | ccgcagctgg acgcccctcc cgggctcact ttgcaacgct gacggtgccg gcagtggccg |
| 301 | tggaggtggg aacagcggcg gcatcctccc ccctggtcac agcccaagcc aggacgcccg |
| 361 | cggaacctct cggctgtgct ctcccatgag tcgggatcgc agcatccccc accagccgct |
| 421 | caccgcctcc gggagccgct gggcttgtac accgcagccc ttccgggaca gcagctgtga |
| 481 | ctccccccca gtgcagattt cgggacagct ctctagaaac tcgctctaaa gacggaaccg |
| 541 | ccacagcact caaagcccac tgcggaagag ggcagcccgg caagcccggg ccctgagcct |
| 601 | ggacccttag cggtgccggg cagcactgcc ggcgcttcgc ctcgccggac gtccgctcct |
| 661 | cctacactct cagcctccgc tggagagacc cccagcccca ccattcagcg cgcaagatac |
| 721 | cctgcagata tgccctgcgt ccaagcccaa tatagccctt cccctccagg ttccagttat |
| 781 | gcggcgcaga catacagctc ggaatacacc acggagatca tgaaccccga ctacaccaag |
| 841 | ctgaccatgg accttggcag cactgagatc acggctacag ccaccacgtc cctgcccagc |
| 901 | atcagtacct tcgtggaggg ctactcgagc aactacgaac tcaagccttc ctgcgtgtac |
| 961 | caaatgcagc ggcccttgat caaagtggag gaggggcggg cgcccagcta ccatcaccat |
| 1021 | caccaccacc accaccacca ccaccaccat caccagcagc agcatcagca gccatccatt |
| 1081 | cctccagcct ccagcccgga ggacgaggtg ctgcccagca cctccatgta cttcaagcag |
| 1141 | tccccaccgt ocaccoccac cacgccggcc ttccccccgc aggcgggggc gttatgggac |
| 1201 | gaggcactgc cctcggcgcc cggctgcatc gcacccggcc cgctgctgga cccgccgatg |
| 1261 | aaggcggtcc ccacggtggc cggcgcgcgc ttcccgctct tccacttcaa gccctcgccg |
| 1321 | ccgcatcccc ccgcgcccag cccggccggc ggccaccacc tcggctacga cccgacggcc |
| 1381 | gctgccgcgc tcagcctgcc gctgggagcc gcagccgccg cgggcagcca ggccgccgcg |
| 1441 | cttgagagcc acccgtacgg gctgccgctg gccaagaggg cggccccgct ggccttcccg |
| 1501 | cctctcggcc tcacgccctc ccctaccgcg tccagcctgc tgggcgagag tcccagcctg |
| 1561 | ccgtcgccgc ccagcaggag ctcgtcgtct ggcgagggca cgtgtgccgt gtgcggggac |
| 1621 | aacgccgcct gccagcacta cggcgtgcga acctgcgagg gctgcaaggg ctttttcaag |
| 1681 | agaacagtgc agaaaaatgc aaaatatgtt tgcctggcaa ataaaaactg cccagtagac |
| 1741 | aagagacgtc gaaaccgatg tcagtactgt cgatttcaga agtgtctcag tgttggaatg |
| 1801 | gtaaaagaag ttgtccgtac agatagtctg aaagggagga gaggtcgtct gccttccaaa |
| 1861 | ccaaagagcc cattacaaca ggaaccttct cagccctctc caccttctcc tccaatctgc |
| 1921 | atgatgaatg cccttgtccg agctttaaca gactcaacac ccagagatct tgattattcc |
| 1981 | agatactgtc ccactgacca ggctgctgca ggcacagatg ctgagcatgt gcaacaattc |
| 2041 | tacaacctcc tgacagcctc cattgatgta tccagaagct gggcagaaaa gattccggga |
| 2101 | tttactgatc tccccaaaga agatcagaca ttacttattg aatcagcctt tttggagctg |
| 2161 | tttgtcctca gactttccat caggtcaaac actgctgaag ataagtttgt gttctgcaat |
| 2221 | ggacttgtcc tgcatcgact tcagtgcctt cgtggatttg gggagtggct cgactctatt |
| 2281 | aaagactttt ccttaaattt gcagagcctg aaccttgata tccaagcctt agcctgcctg |
| 2341 | tcagcactga gcatgatcac agaaagacat gggttaaaag aaccaaagag agtcgaagag |
| 2401 | ctatgcaaca agatcacaag cagtttaaaa gaccaccaga gtaagggaca ggctctggag |
| 2461 | cccaccgagt ccaaggtcct gggtgccctg gtagaactga ggaagatctg caccctgggc |
| 2521 | ctccagcgca tcttctacct gaagctggaa gacttggtgt ctccaccttc catcattgac |
| 2581 | aagctcttcc tggacaccct acctttctaa tcaggagcag tggagcagtg agctgcctcc |
| 2641 | tctcctagca cctgcttgct acgcagcaaa gggataggtt tggaaaccta tcatttcctg |
| 2701 | tccttcctta agaggaaaag cagctcctgt agaaagcaaa gactttcttt tttttctggc |
| 2761 | tcttttcctt acaacctaaa gccagaaaac ttgcagagta ttgtgttggg gttgtgtttt |
| 2821 | atatttaggc attgggggat ggggtgggag ggggttatag ttcatgaggg ttttctaaga |
| 2881 | aattgctaac aaagcacttt tggacaatgc tatcccagca ggaaaaaaaa ggataatata |
| 2941 | actgttttaa aactctttct ggggaatcca attatagttg ctttgtattt aaaaacaaga |
| 3001 | acagccaagg gttgttcgcc agggtaggat gtgtcttaaa gattggtccc ttgaaaatat |
| 3061 | gcttcctgta tcaaaggtac gtatgtggtg caaacaaggc agaaacttcc ttttaatttc |
| 3121 | cttcttcctt tattttaaca aatggtgaaa gatggaggat tacctacaaa tcagacatgg |
| 3181 | caaaacaata atggctgttt gcttccataa acaagtgcaa ttttttaaag tgctgtctta |
| 3241 | ctaagtcttg tttattaact ctcctttatt ctatatggaa ataaaaagga ggcagtcatg |
| 3301 | ttagcaaatg acacgttaat atccctagca gaggctgtgt tcaccttccc tgtcgatccc |
| 3361 | ttctgaggta tggcccatcc aagactttta ggccattctt gatggaacca gatccctgcc |
| 3421 | ctgactgtcc agctatcctg aaagtggatc agattataaa ctggattaca tgtaactgtt |
| 3481 | ttggttgtgt tctatcaacc ccaccagagt tccctaaact tgcttcagtt atagtaactg |
| 3541 | actggtatat tcattcagaa gcgccataag tcagttgagt atttgatccc tagataagaa |
| 3601 | catgcaaatc agcaggaact ggtcatacag ggtaagcacc agggacaata aggattttta |
| 3661 | tagatataat ttaatttttg ttattggtta aggagacaat tttggagagc aagcaaatct |
| 3721 | ttttaaaaaa tagtatgaat gtgaatacta gaaaagattt aaaaaatagt atgagtgtga |
| 3781 | gtactaggaa ggattagtgg gctgcgtttc aacattccgt gttcgtactc ccttttgtat |
| 3841 | gtttctactg ttaatgccat attactatga gataatttgt tgcatagtgt ccttatttgt |
| 3901 | ataaacattt gtatgcacgt tatattgtaa tagctttgcc tgtatttatt gcaagaccac |
| 3961 | cagctcctgg aagctgagtt acagagtaat taaatggggt gttcacagtg acttggatac |
| 4021 | accaattaga aattaaataa gcaaatatat atatatatat aaatatagca ggttacatat |
| 4081 | atatatttat aatgtgtctt tttattaacc atttgtacaa taaatgtcac ttcccatgcc |
| 4141 | gttattttat ggttcatttg cagtgacttt taaggcagta ctgtttagca ctttgatatt |
| 4201 | aaaattttgc ttatgttttg ctaaattcga ataatgtttg aagattttta ggtctaaaag |
| 4261 | tctttatatt atatactctg tatcaagtca aaatatcttt ggccattttg ctaagaaaca |
| 4321 | aactttgaat gtcaaactga tgtcacagta gtttttgtta gctttaaatc atttttgctt |
| 4381 | tagtcttttt aaaggaaaat aacaaaacta tgctgtttat attgtcatta aattatacaa |
| 4441 | tcaaacaaat gccaaatgaa ttgcctaatt gctgcaaagt ataacccaga taggaaatca |
| 4501 | tatgtttttt tccaagagtc attctaatat ttgattatgt tatgtgtgct tttatgaaag |
| 4561 | attgttattt ttatatatca agatgataga acctggaatg ttaggatttt gaaatgttag |
| 4621 | acttggaagg ggcctggtct gtcaactagt ccaacccctt aaaattcata gaggagcaaa |
| 4681 | ctggggccca ttgaagggtg aagagttact caaggtcaaa cagctggtaa cagaatcaag |
| 4741 | actaagacct aatttacctt tccatactct ttttttttct caacttcatc tatataaaat |
| 4801 | caggctttta aacataacca ctaatattta cctgaagata accatgagta aagtatactt |
| 4861 | ttgcattaat tttttgagct tatatgcaaa cataataaat attattaaat atcaggaaag |
| 4921 | ctaacatttc atacaagata gcttcagacc aaattcaaat tgaatttgaa taaattagaa |
| 4981 | atactgtgca tacataacct tcttgtgcac catgagtatt tggaaagtta atccttgttt |
| 5041 | ttgtcgtgtc tataaaggaa gaacaaaaca aaataaaaac agagccctag agaaatgctg |
| 5101 | ttacttttta tttttacacc catcagattt aaggaaaaga ctttttagcc attataatct |
| 5161 | agtggttgga aggaatgaag aagctttttt agtaataggt ccagatatga gtgctaaaaa |
| 5221 | taaagatgat agcatgttct tctgtcttcc atagttatta caactatgag agcctcccaa |
| 5281 | gtcatcttat caactcaact cccttttttt tgtcttaatg ttgcacataa gtttatacag |
| 5341 | agtggatgac cacactagca cagaagagaa caacatgtat taaagcaggt gattcctccc |
| 5401 | cttggcggga gagctctctc agtgtgaaca tgccttctgt gggcggaaat caggaagcca |
| 5461 | ccagctgtta atggagagtg ccttgctttt atttcagaca gcagagtttt ccaaagtttc |
| 5521 | tctgctcctc taacagcatt gctctttagt gtgtgttaac ctgtggtttg aaagaaatgc |
| 5581 | tcttgtacat taacaatgta aatttaaatg attaaattac attttatcaa tggca |

1. The use of an agonist of a member of the nuclear hormone receptor NR4A subgroup in the manufacture of a medicament for the prevention, reduction or inhibition of scarring in a non-ocular tissue.

2-18. (canceled)

19. A method of preventing, reducing or inhibiting scarring in a non-ocular tissue, the method comprising administering a therapeutically effective amount of an agonist of a member of the nuclear hormone receptor NR4A subgroup to a patient in need of such prevention, reduction or inhibition.

20-22. (canceled)
